(19) United States
(12) Patent Application Publication
(10) Pub. No.: US 2015/0374452 A1
(43) Pub. Date: Dec. 31, 2015

(54) PROJECTION SYSTEM

(71) Applicant: Panasonic Intellectual Property Management Co., Ltd., Osaka (JP)

(72) Inventor: Tomoyuki SAITO, Osaka (JP)

(21) Appl. No.: 14/741,488

(22) Filed: Jun. 17, 2015

(30) Foreign Application Priority Data
Jun. 25, 2014 (JP) 2014-130002
Mar. 11, 2015 (JP) 2015-048852

(51) Int. Cl. A61B 19/00; A61B 5/00

(52) U.S. Cl. CPC A61B 19/5244; A61B 5/0071; A61B 5/746; A61B 5/0075; A61B 2019/5257; A61B 2019/5293; A61B 2560/0475

(57) ABSTRACT

A projection system includes an imaging unit, a projector, a distance detector, and a controller. The imaging unit captures an image of an object. The projector generates a projection image based on the captured image to project the projection image onto the object. The distance detector detects a distance to the object. The controller controls operations of the imaging unit and the projector. The controller determines whether the distance detected by the distance detector falls within a predetermined interval range. When the detected distance falls within the predetermined interval range, the controller causes the projector to project the projection image onto the object based on the captured image.

### BACKGROUND

1. Field

The present disclosure relates to a projection system that projects an image onto a physical body.

2. Description of the Related Art

For example, in a surgical operation support system disclosed in Unexamined Japanese Patent Publication No. 9-24053, a fluorescent imaging device outputs image data indicating an affected part of a living body to be given surgery, and an image projection device plays back an image of the image data to display the image on the actual affected part. A substance that emits fluorescence by irradiation of the substance with light having a predetermined wavelength is previously injected in the affected part of the living body. That is, the surgical operation support system supports confirmation of an affected region by displaying, on the actual affected part, a fluorescent image showing fluorescence emitted from the affected part.

### SUMMARY

According to one aspect of the present disclosure, a projection system includes an imaging unit, a projector, a distance detector, and a controller. The imaging unit captures an image of an object. The projector generates a projection image based on the captured image to project the projection image onto the object. The distance detector detects a distance to the object. The controller controls operations of the imaging unit and the projector. The controller determines whether the distance detected by the distance detector falls within a predetermined interval range. When the detected distance falls within the predetermined interval range, the controller causes the projector to project the projection image onto the object based on the captured image.

### BRIEF DESCRIPTION OF DRAWINGS

FIG. 1 is a schematic diagram illustrating a configuration of a surgery support system;

FIG. 2A illustrates a state of a surgical field in the surgery support system before a projection operation is performed;

FIG. 2B illustrates a state in which the projection operation is performed on the surgical field in FIG. 2A;

FIG. 3 is a schematic diagram illustrating a configuration of a deviation adjustment system;

FIG. 4A is a perspective view illustrating an appearance of a light adjustment device;

FIG. 4B is an exploded perspective view illustrating a configuration of the light adjustment device;

FIG. 5A is a perspective view illustrating the light adjustment device during deviation adjustment;

FIG. 5B illustrates an example in a state of a projection surface when the deviation is unadjusted;

FIG. 5C illustrates an image for projection in the example of FIG. 5B;

FIG. 5D illustrates the image for projection in which the deviation of the image in FIG. 5C is adjusted;

FIG. 5E illustrates an example in a state of the projection surface after the deviation is adjusted;

FIG. 6 illustrates a state of the projection surface in a use example of the light adjustment device;

FIG. 7A is a plan view of an opening mask′ in an application example;

FIG. 7B illustrates a state in which a fluorescent image is projected onto a projection surface using the opening mask in FIG. 7A;

FIG. 7C illustrates a state in which a projection operation of the surgery support system is performed on the projection surface in FIG. 7B;

FIG. 8A is a view illustrating an infrared fluorescence and a visible laser beam before the deviation is adjusted;

FIG. 8B is a view illustrating the infrared fluorescence and the visible laser beam after the deviation is adjusted;

FIG. 9 is a view illustrating a scan pattern of a projector;

FIG. 10 is a flowchart illustrating a cutting auxiliary line projecting operation according to detection of an affected part;

FIG. 11A is a view illustrating the cutting auxiliary line projecting operation in a first cutting margin width;

FIG. 11B is a view illustrating the cutting auxiliary line projecting operation in a second cutting margin width;

FIG. 12A illustrates a state of a conventional surgery;

FIG. 12B is a view illustrating projection of surgical auxiliary information to a surrounding of the affected part;

FIG. 13A is a top view illustrating an auxiliary screen member in a state in which the surgical auxiliary information is not projected;

FIG. 13B is a top view illustrating the auxiliary screen member in a state in which the surgical auxiliary information is projected;

FIG. 14 is a flowchart illustrating a processing flow in a use height monitoring operation.

FIG. 15A is a view illustrating the monitoring operation when a distance falls within an acceptable range of the use height;

FIG. 15B is a view illustrating the monitoring operation when the distance is outside the acceptable range of the use height; and

FIG. 16 is a timing chart illustrating operations of an infrared excitation light source, a TOF sensor, and a visible-light laser.

### DETAILED DESCRIPTION

Hereinafter, an exemplary embodiment will be described in detail with reference to the drawings as appropriate. However, unnecessarily detailed description may occasionally be omitted. For example, detailed description of well-known matters and redundant description of substantially the same configurations may occasionally be omitted. The omission of these items is to avoid the following description from becoming unnecessarily redundant, and to ease understanding of those skilled in the art.

The applicant provides the accompanying drawings and the following description in order that those skilled in the art sufficiently understand the present disclosure, and it is not intended to limit subjects described in claims by the accompanying drawings and the following description.

### First Exemplary Embodiment

### 1. Outline of Surgery Support System

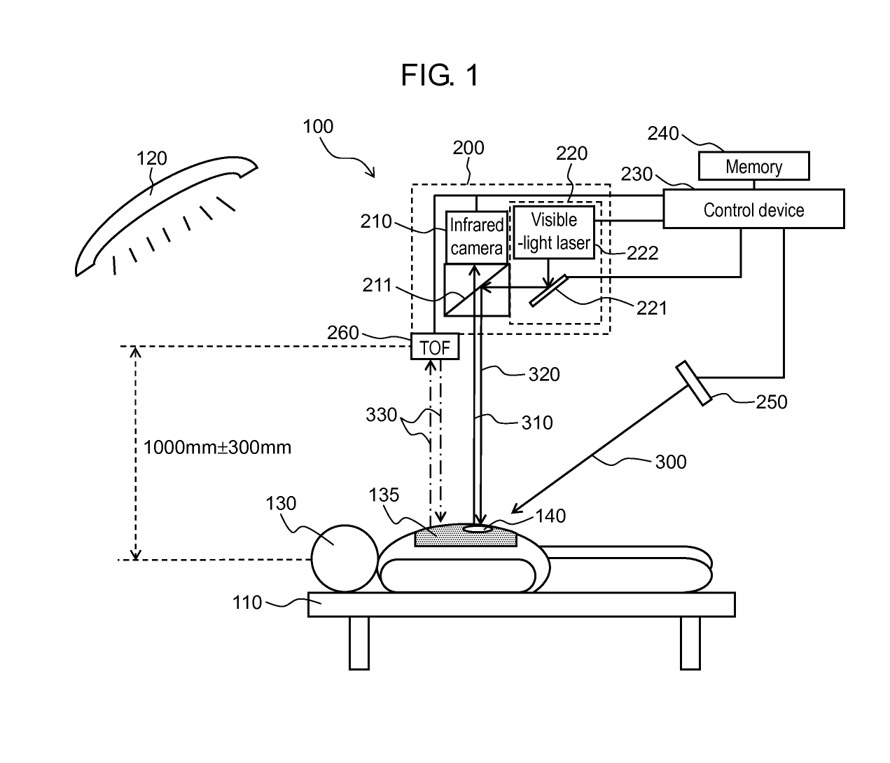

Referring to FIG. 1, an outline of a surgery support system according to a first exemplary embodiment will be described as an example of a projection system of the present disclosure. FIG. 1 is a schematic diagram illustrating a configuration of surgery support system 100 of the first exemplary embodiment.

Using a projection image, surgery support system 100 visually supports a surgery performed on a patient by a doctor and the like in a surgery room, for example. For use of surgery support system 100, a photosensitive substance is injected into a blood of patient 130 to be given surgery.

The photosensitive substance reacts with excitation light to yield fluorescence. In the first exemplary embodiment, indocyanine green (hereinafter, referred to as “ICG”) is used as an example of the photosensitive substance. ICG is certificated as a medical substance, and is a reagent that can be used in a human body. ICG generates infrared fluorescence around a peak wavelength of 850 nm when being irradiated with infrared excitation light around a wavelength of 800 nm. When injected into the blood, ICG is accumulated in affected part 140 in which a flow of the blood or a lymph fluid is delayed. Therefore, a region of affected part 140 can be specified by detecting an infrared fluorescence region generating the infrared fluorescence.

Because the infrared fluorescence generated by the region of affected part 140 is non-visible light, the doctor and the like can hardly directly specify the region of affected part 140 when visually observing surgical field 135. For this reason, surgery support system 100 detects the region generating the infrared fluorescence of ICG to specify the region of affected part 140. Surgery support system 100 irradiates the specific region of affected part 140 with visible light such that a human can visually observe the specific region of affected part 140. Accordingly, a projection image visualizing the specific region of affected part 140 is projected, which allows surgery support system 100 to support the specification of the region of affected part 140 performed by the doctor or the like who performs the surgery.

### 2. Configuration of Surgery Support System

A configuration of surgery support system 100 will be described below with reference to FIG. 1. Surgery support system 100 is placed and used in a surgery room of a hospital. Surgery support system 100 includes imaging and irradiation device 200, control device 230, memory 240, and infrared excitation light source 250. Although not illustrated, surgery support system 100 includes a mechanism that changes a placement position of imaging and irradiation device 200. For example, the mechanism includes a driving arm that is mechanically connected to imaging and irradiation device 200, and a caster of a base on which a set of surgery support system 100 is placed.

Imaging and irradiation device 200 integrally includes an imaging unit and an irradiation unit. Imaging and irradiation device 200 includes infrared camera 210, dichroic mirror 211, projector 220, and TOF (Time-of-Flight) sensor 260. Projector 220 includes visible-light laser 222 and a MEMS (Micro Electro Mechanical System) mirror 221.

Control device 230 provided in a controller totally controls each of units constituting surgery support system 100. Control device 230 is electrically connected to infrared camera 210, visible-light laser 222, MEMS mirror 221, TOF sensor 260, memory 240, and infrared excitation light source 250, and outputs a control signal to control each unit. For example, control device 230 includes a CPU or an MPU, and implements a function by executing a predetermined program. Alternatively, the function of control device 230 may be implemented by a specifically-designed electronic circuit or a reconfigurable electronic circuit (such as an ASIC and an FPGA).

For example, memory 240 includes a ROM (Read Only Memory) or a RAM (Random Access Memory). Memory 240 is a storage medium that is properly accessed by control device 230 in performing various calculations.

Infrared excitation light source 250 emits infrared excitation light 300 having at least a spectrum including a wavelength band component around the ICG excitation wavelength of 800 nm. Infrared excitation light source 250 can turn on and off of irradiation of infrared excitation light 300 in response to a control signal from control device 230. In the example of FIG. 1, infrared excitation light source 250 is disposed outside imaging and irradiation device 200, but the present disclosure is not limited thereto. Alternatively, infrared excitation light source 250 may be disposed inside imaging and irradiation device 200 as long as an irradiation port for emitting the infrared excitation light is properly provided.

A configuration of each of units constituting imaging and irradiation device 200 will be described below.

Infrared camera 210 used in the imaging unit has a spectral sensitivity characteristic in which light-receiving sensitivity is high in an infrared region. In surgery support system 100 of the first exemplary embodiment, it is necessary to detect the infrared fluorescence around the wavelength of 850 nm from ICG. Therefore, infrared camera 210 is used having the spectral sensitivity characteristic in which the light-receiving sensitivity is high in the infrared region around the wavelength of 850 nm. A bandpass filter that allows passage of only the light having the wavelength near 850 nm may be disposed in front of an imaging surface of infrared camera 210 in order to restrain the reception of the light other than the infrared fluorescence from ICG. A wavelength spectrum of the infrared fluorescence is an example of a first spectrum. Infrared camera 210 sends the captured image (infrared image) indicating an imaging result to control device 230.

In projector 220, visible-light laser 222 is a laser device that emits visible light. A laser source having any wavelength may be used as visible-light laser 222 as long as a human can visibly recognize the light. Visible-light laser 222 may include only a monochrome laser source, or visible-light laser 222 may include laser sources having a plurality of colors that can be switched in response to a control signal from control device 230. Visible-light laser 222 emits visible laser beam 320 toward MEMS mirror 221.

MEMS mirror 221 is one in which many micro-mirror surfaces are two-dimensionally arrayed. For example, MEMS mirror 221 includes a digital mirror device. In MEMS mirror 221, visible laser beam 320 emitted from visible-light laser 222 is incident on each micro-mirror surface. MEMS mirror 221 generates the visible-light projection image by reflecting visible-light laser 222 in a direction corresponding to a tilt angle of the micro-mirror surface.

At this point, control device 230 horizontally and vertically controls the tilt angle of each micro-mirror surface of MEMS mirror 221. Therefore, control device 230 performs two-dimensional scans in the horizontal and vertical directions with visible laser beam 320 to generate the projection image in projector 220. Visible laser beam 320 reflected by the micro-mirror surface of MEMS mirror 221 reaches dichroic mirror 211.

In the first exemplary embodiment, MEMS mirror 221 is illustrated as a component of projector 220, but the present disclosure is not limited thereto. Alternatively, for example, a galvano mirror may be used. That is, any optical element may be used as long as the optical element can perform the horizontal scan and the vertical scan.

Dichroic mirror 211 is disposed so as to face infrared camera 210 and MEMS mirror 221. Dichroic mirror 211 has a function of transmitting a specific wavelength band component (including the wavelength of 850 nm) in the incident light and of reflecting other wavelength band components (including a visible component). In the first exemplary embodiment, as illustrated in FIG. 1, infrared camera 210 is disposed immediately above dichroic mirror 211 while MEMS mirror 221 is disposed in a horizontal direction of dichroic mirror 211. Because of the above optical characteristic, dichroic mirror 211 reflects visible laser beam 320 emitted from visible-light laser 222, and transmits infrared fluorescence 310 toward the imaging surface of infrared camera 210.

As illustrated in FIG. 1, dichroic mirror 211, projector 220, and infrared camera 210 are aligned such that an optical path of visible laser beam 320 reflected by dichroic mirror 211 is matched with an optical path of infrared fluorescence 310 incident on the imaging surface of infrared camera 210. Therefore, accuracy for the irradiation of visible laser beam 320 can be enhanced with respect to the region emitting infrared fluorescence 310 (affected part 140).

TOF sensor 260 emits infrared detection light 330 to receive infrared detection light 330 reflected by a target object, thereby detecting distance information indicating the distance to the target object. A wavelength spectrum of infrared detection light 330 is an example of a second spectrum. In TOF sensor 260, infrared light having wavelength band of 850 nm to 950 nm is used as infrared detection light 330. The second spectrum can be superimposed on at least a part of the first spectrum. TOF sensor 260 measures the distance to the target object based on a delay time until infrared detection light 330 reflected by the target object is received since infrared detection light 330 is emitted and a speed of light. Alternatively, TOF sensor 260 may measure the distance to the target object based on a difference between a voltage value of infrared detection light 330 during the irradiation and a voltage value of infrared detection light 330 that is received after reflected by the target object. TOF sensor 260 sends distance information on the measured distance to the target object to control device 230.

As illustrated in FIG. 1, in addition to surgery support system 100, surgical table 110 and shadowless lamp 120 are installed in the surgery room. Patient 130 is placed on surgical table 110. Shadowless lamp 120 is a lighting fixture that lights affected part 140 of patient 130 placed on surgical table 110. Shadowless lamp 120 irradiates a working region of the doctor with light having high illuminance (30000 lux to 100000 lux) so as not to form a shadow in the working region.

Surgery support system 100 is disposed such that imaging and irradiation device 200 is located immediately above patient 130 placed on surgical table 110. In surgery support system 100 of the first exemplary embodiment, in order to secure accuracy for region specification of affected part 140 with infrared camera 210, an acceptable range of a use height is defined based on a focal distance fixed from an optical system of infrared camera 210. In the first exemplary embodiment, it is assumed that a height from a body axis of patient 130 placed on surgical table 110 to imaging and irradiation device 200 (TOF sensor 260) in a range of 1000 mm±300 mm is the acceptable range of the use height. The acceptable range of the use height is described in detail later.

### 2. Basic Operation of Surgery Support System

A start-up operation and a projection operation, which are the basic operation of surgery support system 100, will be described below.

### 2-1. Surgery Support System Start-Up Operation

The start-up operation of surgery support system 100 will be described. In surgery support system 100, control device 230 is started up when a power supply (not illustrated) is switched from an off state to an on state. The started-up control device 230 performs the start-up operation of each of units, such as infrared camera 210, visible-light laser 222, infrared excitation light source 250, and TOF sensor 260, which constitute surgery support system 100.

When the start-up operation is performed, visible-light laser 222 starts an operation to amplify visible laser beam 320. Imaging and irradiation device 200 becomes a usable state at the time output of visible laser beam 320 is stabilized.

### 2-2. Basic Projection Operation of Surgery Support System

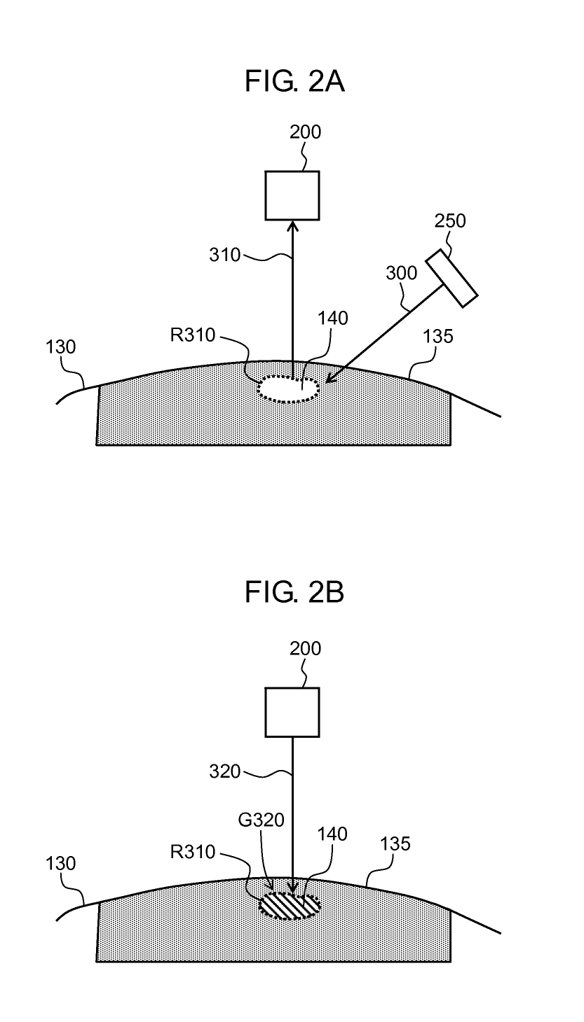

A basic projection operation of surgery support system 100 will be described below with reference to FIGS. 1, 2A, and 2B. FIGS. 2A and 2B are views illustrating a state of surgical field 135 in surgery support system 100 of FIG. 1. FIG. 2A illustrates the state of surgical field 135 in surgery support system 100 before the projection operation is performed. FIG. 2B illustrates the state in which the projection operation is performed on surgical field 135 in FIG. 2A.

In the state of FIG. 2A, control device 230 first drives infrared excitation light source 250 to irradiate surgical field 135 including affected part 140 with infrared excitation light 300. Then, infrared excitation light 300 excites ICG deposited on affected part 140 in surgical field 135, which allows affected part 140 to emit infrared fluorescence 310.

Infrared camera 210 captures the image of affected part 140 in surgical field 135 under the control of control device 230. At this point, the captured image includes an image of infrared fluorescence region R310 emitting infrared fluorescence 310. Infrared camera 210 sends the captured image to control device 230.

Control device 230 detects infrared fluorescence region R310 based on the captured image sent from infrared camera 210. Specifically, control device 230 acquires information indicating a coordinate of infrared fluorescence region R310 in the captured image by calculating an XY-coordinate from one vertex of the captured image.

Memory 240 stores information indicating a correspondence relationship between a coordinate in the captured image of infrared camera 210 and a coordinate in data used to generate the projection image by MEMS mirror 221. Based on the information indicating the correspondence relationship stored in memory 240, control device 230 controls MEMS mirror 221 such that the coordinate corresponding to the acquired coordinate is irradiated with visible laser beam 320. Projector 220 is controlled so as to scan and irradiate the coordinate with visible laser beam 320.

As illustrated in FIG. 2B, projection image G320 of visible laser beam 320 is projected onto infrared fluorescence region R310 in surgical field 135 by the irradiation with visible laser beam 320. Thus, in surgery support system 100, the region of affected part 140 emitting non-visible infrared fluorescence 310 is specified by detecting infrared fluorescence region R310 based on the captured image of infrared camera 210. Additionally, projector 220 properly projects projection image G320, which allows the visualization of the region of affected part 140 that cannot directly visually be recognized in the surgical field. For example, projection image G320 is a monochrome, uniform image with visible-light laser 222.

The above processing is repeatedly performed in a predetermined cycle (for example, 1/60 second). Therefore, for example, the image captured every 1/60 second is projected, and the doctor and the like can visually recognize the position and shape of affected part 140 in real time.

### 3. Projection Deviation Adjustment Method in Surgery Support System

### 3-1. Outline of Projection Deviation Adjustment Method

In surgery support system 100, as described above, affected part 140 emitting invisible infrared fluorescence 310 of ICG is detected with infrared camera 210 (refer to FIG. 2A), the projection image of visible laser beam 320 is projected, and affected part 140 is visualized by projection image G320 (refer to FIG. 2B). When projection image G320 is projected during the use of surgery support system 100 while deviating from infrared fluorescence region R310 of affected part 140, an error occurs with respect to the position of affected part 140 in surgical field 135. Therefore, a relationship between a position specified based on the captured image of infrared camera 210 and a projection position of the projection image is checked before the use of surgery support system 100, and it is necessary to adjust surgery support system 100 when the position deviation is generated.

Work to check and adjust the position deviation is performed in various scenes before the use of surgery support system 100. For example, when the placement position is fixed in imaging and irradiation device 200, the adjustment work is performed at a manufacturing stage such that visible-light laser 222 irradiates the region specified by infrared camera 210 with visible laser beam 320. The adjustment work is also performed at assembly stage of imaging and irradiation device 200, because an error slightly occurs between the irradiation position of visible-light laser 222 and the imaging position of infrared camera 210. A post-assembly disturbance and a difference in angle of view between infrared camera 210 and projector 220 also cause the position deviation. Because security assurance has a higher priority in medical use, it is necessary to successively check the position deviation before the start of the surgery in which surgery support system 100 is used.

In the present disclosure, the imaging target of infrared camera 210 is easily visualized, and the position deviation of the projection image can visually be recognized. The deviation between the irradiation position of visible-light laser 222 and the imaging position of infrared camera 210 can easily be adjusted by the method for adjusting the position deviation with the light adjustment device.

The configuration of the light adjustment device and the method for adjusting the deviation with the light adjustment device will sequentially be described below.

### 3-2. Configurations of Deviation Adjustment System and Light Adjustment Device

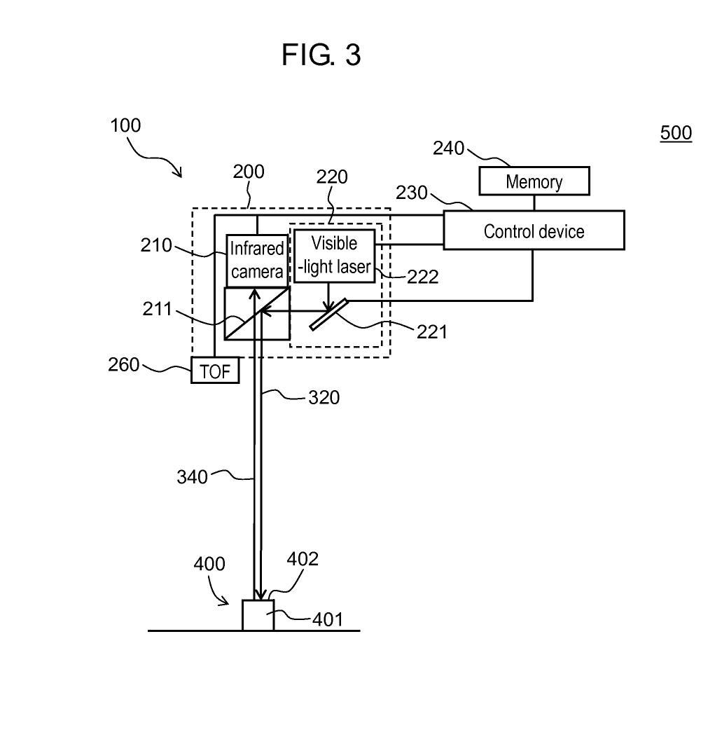

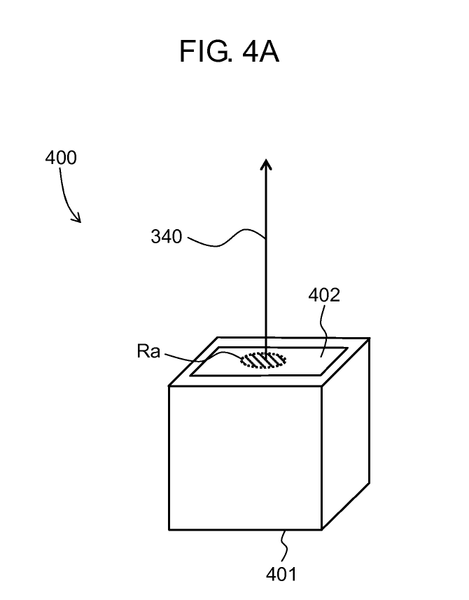

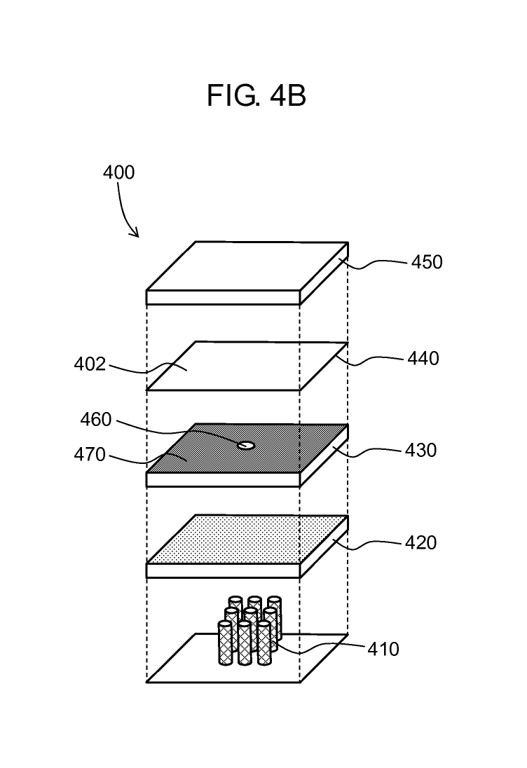

A configuration of the light adjustment device will be described below with reference to FIGS. 3, 4A, and 4B. FIG. 3 is a schematic diagram illustrating the configuration of deviation adjustment system 500 that adjusts a deviation between an irradiation position of visible-light laser 222 and an imaging position of infrared camera 210. FIGS. 4A and 4B are views illustrating the configuration of light adjustment device 400. FIG. 4A is a perspective view illustrating an appearance of light adjustment device 400. FIG. 4B is an exploded perspective view illustrating the configuration of light adjustment device 400.

Deviation adjustment system 500 includes surgery support system 100 and light adjustment device (light source device) 400. Deviation adjustment system 500 is an example of the projection system. FIG. 3 illustrates a placement state of light adjustment device 400 with respect to surgery support system 100 in deviation adjustment system 500. As illustrated in FIG. 3, light adjustment device 400 includes projection surface 402 that is a target of imaging and projection operations of surgery support system 100 and a light source that is located in chassis 401. Projection surface 402 is one of surfaces of box-shaped chassis 401. As illustrated in FIG. 4A, projection surface 402 of light adjustment device 400 is also used as an output surface of LED (Light Emitting Diode) light 340 output from the inside of chassis 401. FIG. 4B illustrates an internal structure of a chassis 401 of light adjustment device 400. As illustrated in FIG. 4B, light adjustment device 400 includes white LED 410, diffuser plate 420, opening mask 430, screen member 440, and protective glass 450. Chassis 401 of light adjustment device 400 has the internal structure in which white LED 410, diffuser plate 420, opening mask 430, screen member 440, and protective glass 450 are sequentially overlapped.

White LED 410 is a semiconductor light emitting element that emits white LED light 340. The wavelength spectrum of the light emitted from white LED 410 includes not only the visible region but also the non-visible region (including infrared region). In the present disclosure, white LED 410 is used as the light source of light adjustment device 400, but the present disclosure is not limited thereto. Alternatively, a light source having a spectrum including the visible component and the non-visible component (including the infrared wavelength component) may be used instead of white LED 410. For example, both a light emitting element, such as a monochrome LED, which emits only the visible light and a light emitting element that emits only the infrared light may be disposed in chassis 401 to constitute the light source. Any light source that can coaxially emit the visible light and the infrared light may be used.

For example, diffuser plate 420 is constructed with a resin plate having a ground-glassy rough surface. Diffuser plate 420 is disposed in chassis 401 such that diffuser plate 420 faces white LED 410. Diffuser plate 420 performs surface emission by reducing luminance unevenness of the light emitted from white LED 410. Light adjustment device 400 may not necessarily include diffuser plate 420.

Opening mask 430 is a light shielding member in which opening 460 is provided in light shielding surface 470. Opening mask 430 is disposed in chassis 401 of light adjustment device 400 such that opening mask 430 faces white LED 410 with diffuser plate 420 interposed therebetween. Opening 460 having a predetermined size faces white LED 410, and the light emitted from white LED 410 passes through opening 460. Light shielding surface 470 surrounds opening 460 to shield the light incident from white LED 410. The size of opening 460 and the position of opening 460 on light shielding surface 470 in opening mask 430 are fixed according to a measurement purpose. For example, opening 460 having the size of 2 mm or less is formed in opening mask 430 in order to check whether the deviation is less than or equal to 2 mm.

Screen member 440 is a light scattering sheet member, and includes projection surface 402 in one of principal surfaces. In screen member 440, the principal surface that is not projection surface 402 is oriented toward opening mask 430. Screen member 440 is disposed facing opening mask 430. At least the visible light component of the light emitted from white LED 410 is scattered by screen member 440. Therefore, as illustrated in FIG. 4A, the light is emitted from white LED 410, and a view angle of reference region Ra that is the output region is expanded, which enables a human to easily perform the visual recognition. Reference region Ra irradiated with white LED 410 is formed with the size corresponding to a setting of opening 460 to constitute a reference for the visual recognition of the position deviation in the deviation adjustment method (to be described later).

For example, screen member 440 is made of paper. Any paper color may be used, and a color that facilitates the visual recognition (for example, complimentary color) may be used according to a color of the emitted laser beam. Alternatively, screen member 440 may be made of cloth instead of paper. Alternatively, screen member 440 may be made of any material, which scatters at least one visible light component in the incident light and has a small scattering rate of the infrared wavelength component.

Protective glass 450 protects screen member 440 from a flaw. Light adjustment device 400 may not necessarily include screen member 440 and protective glass 450.

3-3. Deviation Adjustment Method with Light Adjustment Device

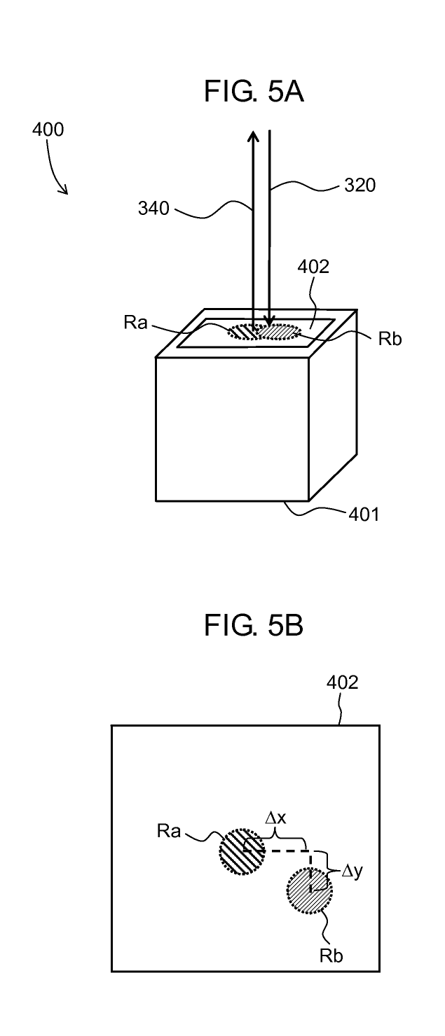

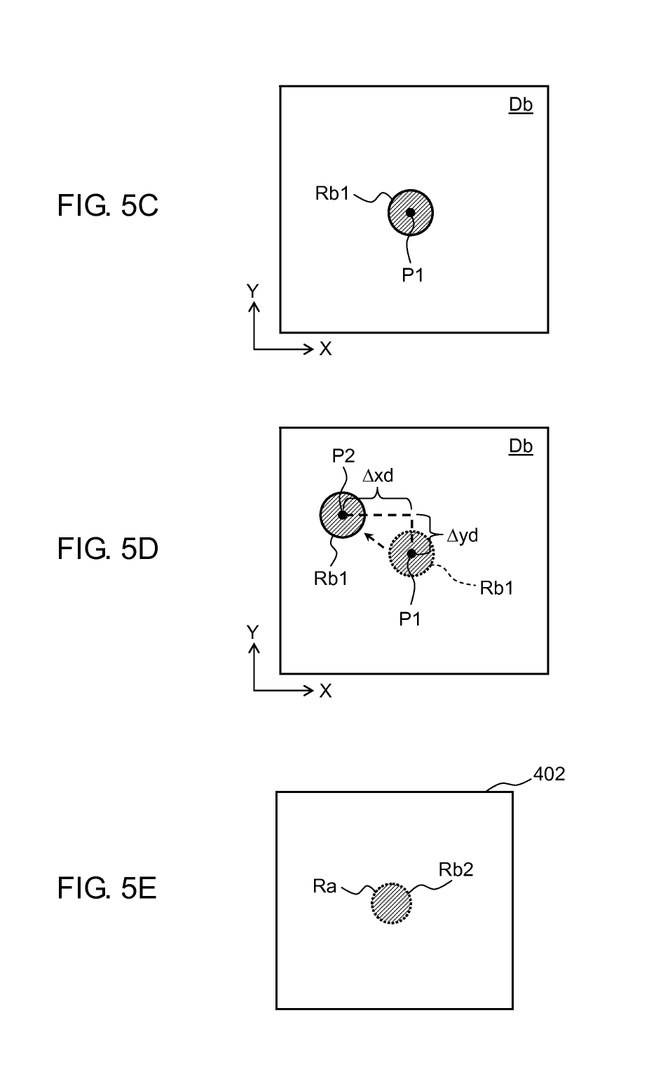

The deviation adjustment method with light adjustment device 400 will be described below with reference to FIGS. 3 and 5A to 5E. FIGS. 5A to 5E are views illustrating the deviation adjustment with light adjustment device 400. FIG. 5A is a perspective view illustrating light adjustment device 400 during the deviation adjustment. FIG. 5B illustrates an example in the state of projection surface 402 when the deviation is unadjusted; FIG. 5C illustrates an image for projection in the example of FIG. 5B. FIG. 5D illustrates the image for projection in which the deviation of the image in FIG. 5C is adjusted. FIG. 5E illustrates an example in the state of projection surface 402 after the deviation is adjusted.

For example, an adjustment worker of a manufacturer performs the adjustment method as adjustment work at a manufacturing stage of imaging and irradiation device 200 or surgery support system 100. In this case, a shipment of imaging and irradiation device 200 or surgery support system 100 is already adjusted. The adjustment method may be performed as confirmation work immediately before the actual surgery even if the adjustment is already performed at the manufacturing stage.

In performing the adjustment method, as illustrated in FIG. 3, the adjustment worker disposes light adjustment device 400 at the position facing the imaging surface of infrared camera 210 and the irradiation port of visible laser beam 320, the position being located directly below imaging and irradiation device 200. At this point, for example, in the case that a height acceptable range that is an acceptable range of the distance (height) between imaging and irradiation device 200 and surgical table 110 is set to 1000 mm±300 mm, light adjustment device 400 is disposed at the position where the distance from the bottom surface of imaging and irradiation device 200 is 1000 mm.

After placing light adjustment device 400, the adjustment worker irradiates light adjustment device 400 with LED light 340 emitted from white LED 410.

LED light 340 is incident on screen member 440 through opening mask 430, and output from reference region Ra in projection surface 402. In screen member 440, the visible light component of LED light 340 generates scattering light. The scattering light of the visible light component of LED light 340 forms an image indicating reference region Ra (hereinafter, referred to as a “reference region image” Ra) on projection surface 402 (refer to FIGS. 4A and 4B).

LED light 340 emitted from white LED 410 includes the wavelength band component in the infrared region. The infrared wavelength band component in LED light 340 is transmitted through dichroic mirror 211 of surgery support system 100.

Surgery support system 100 performs the projection operation on projection surface 402 of light adjustment device 400 which is a target of the imaging and projection. In surgery support system 100, infrared camera 210 receives the light transmitted through dichroic mirror 211 to capture the image of projection surface 402. Therefore, infrared camera 210 captures the image of reference region Ra emitting the light including the infrared wavelength band component. Infrared camera 210 sends the captured image to control device (controller) 230.

Based on the captured image sent from infrared camera 210, control device 230 acquires information indicating the coordinate of reference region image Ra emitting the light in the infrared wavelength band by calculating, for example, the XY-coordinate from one vertex of the captured image. For example, control device 230 manages the coordinate in the captured image sent from infrared camera 210 and a scan coordinate of the irradiation with visible laser beam 320 on one-on-one level on the image data. Control device 230 controls MEMS mirror 221 such that the scan coordinate corresponding to the acquired coordinate is irradiated with the visible laser beam.

Projector 220 irradiates light adjustment device 400 with visible laser beam 320 according to the infrared emission from light adjustment device 400, thereby projecting projection image Rb onto projection surface 402 as illustrated in FIG. 5A. As a result, reference region image Ra that is the imaging target of imaging and irradiation device 200 and projection image Rb of imaging and irradiation device 200 are projected in the visible light onto projection surface 402 of light adjustment device 400, and the adjustment worker can visibly recognize reference region image Ra and projection image Rb.

At this point, originally a projection region onto which reference region image Ra in LED light 340 is projected and a projection region onto which projection image Rb in visible laser beam 320 is projected should be matched with each other. However, actually due to the assembly error and the like, sometimes the deviation is generated between the positions of reference region image Ra and projection image Rb. In such cases, light adjustment device 400 allows the visualization of position deviations Δx and Δy between the positions of reference region image Ra and projection image Rb as illustrated in FIG. 5B.

The adjustment work performed on position deviations Δx and Δy in FIG. 5B will be described below.

Initially, control device 230 stores information indicating the irradiation position (that is, the scan position of MEMS mirror 221) with visible laser beam 320 during the unadjusted deviation, in memory 240. At this point, as illustrated in FIG. 5C, control device 230 generates a video signal indicating image Db in which projection image Rb1 is disposed based on the imaging result of reference region image Ra. Although projection image Rb of visible laser beam 320 is projected onto projection surface 402 based on the video signal, projection image Rb is projected to the position deviating from reference region image Ra as illustrated in FIG. 5B. Control device 230 stores position P1 of projection image Rb1 on image Db during the unadjusted deviation, in memory 240. Hereinafter, the position (scan position) P1 is referred to as an “unadjusted position”.

The adjustment worker compares reference region image Ra and projection image Rb, which are projected onto projection surface 402, to each other while visually viewing reference region image Ra and projection image Rb, and inputs a shift amount to control device 230 using a manipulation unit (not illustrated) such that the positions of reference region image Ra and projection image Rb are matched with each other. Specifically, information on a moving amount for shifting the projection image on an X-axis or a Y-axis is input to control device 230.

Control device 230 controls projector 220 such that the irradiation position (the scan position of MEMS mirror 221) with visible laser beam 320 is changed based on the input information. For example, based on the input information indicating the moving amount, control device 230 shifts the irradiation position on image Db from unadjusted position P1 by moving amounts Δxd and Δyd indicated by the input information as illustrated in the FIG. 5D. Moving amounts Δxd and Δyd on the image are values corresponding to actual position deviation amounts Δx and Δy on projection surface 402. Through the adjustment, as illustrated in FIG. 5E, projection image Rb2 is projected to the position on projection surface 402 corresponding to post-adjustment irradiation position P2, and thus projection image Rb2 is matched with reference region image Ra.

For example, the above work is performed until the adjustment worker determines that reference region image Ra and projection image Rb2, which are projected onto projection surface 402, are matched with each other.

In completing the adjustment work, control device 230 stores final irradiation position P2 (that is, the scan position of MEMS mirror 221) on image Db, in memory 240. Hereinafter, the irradiation position (scan position) P2 is referred to as an “adjusted position”.

Control device 230 calculates a deviation correction amount based on unadjusted position P1 and adjusted position P2, which are stored in memory 240. Specifically, a difference between unadjusted position P1 and adjusted position P2 is calculated as the deviation correction amount. In the example of FIGS. 5B to 5E, moving amounts Δxd and Δyd are stored in memory 240 as the deviation correction amount.

After the deviation is adjusted, control device 230 corrects the irradiation position of visible laser beam 320 based on the deviation correction amount stored in memory 240, and projects the projection image. Therefore, the projection image is accurately projected on the projection target.

### 3-4. Application Example of Light Adjustment Device 400

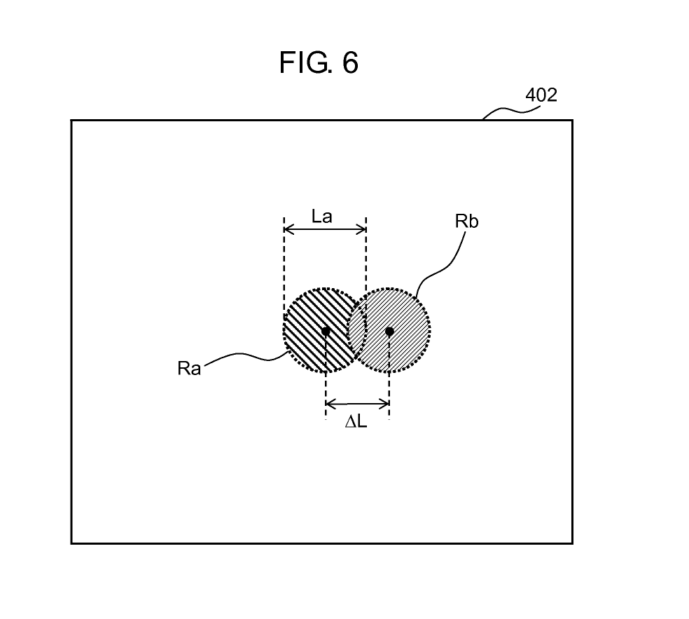

Whether the position deviation amount falls within an acceptable error range can be checked from the reference region image and the projection image, which are projected onto light adjustment device 400. An acceptable error checking method will be described below with reference to FIG. 6. FIG. 6 illustrates an example in the state of projection surface 402 when light adjustment device 400 is used in the location illustrated in FIG. 3.

It is assumed that diameter La of circular reference region image Ra in FIG. 6 is matched with a predetermined acceptable error in a specification of surgery support system 100. Diameter La is set by the size of opening 460 of opening mask 430 (refer to FIGS. 4A and 4B). For example, diameter La is set to 2 mm in the case that the specification of surgery support system 100 requires projection accuracy of the acceptable error of 2 mm. In the example of FIG. 6, it is assumed that the projection magnification of projection image Rb has no error.

As illustrated in FIG. 6, in the case that reference region image Ra and projection image Rb partially overlap each other, position deviation ΔL between reference region image Ra and projection image Rb is less than or equal to diameter La. Therefore, the projection accuracy of surgery support system 100 falls within the acceptable error range. On the other hand, in the case that reference region image Ra and projection image Rb do not overlap each other, because position deviation ΔL is larger than diameter La, the projection accuracy of surgery support system 100 is outside the acceptable error range. Therefore, a user of light adjustment device 400 can easily check whether the position deviation falls within the acceptable error range by visually recognizing whether at least parts of reference region image Ra and projection image Rb overlap each other. In the case that the position deviation falls within the acceptable error range, the manipulation of control device 230 for the deviation adjustment may be omitted.

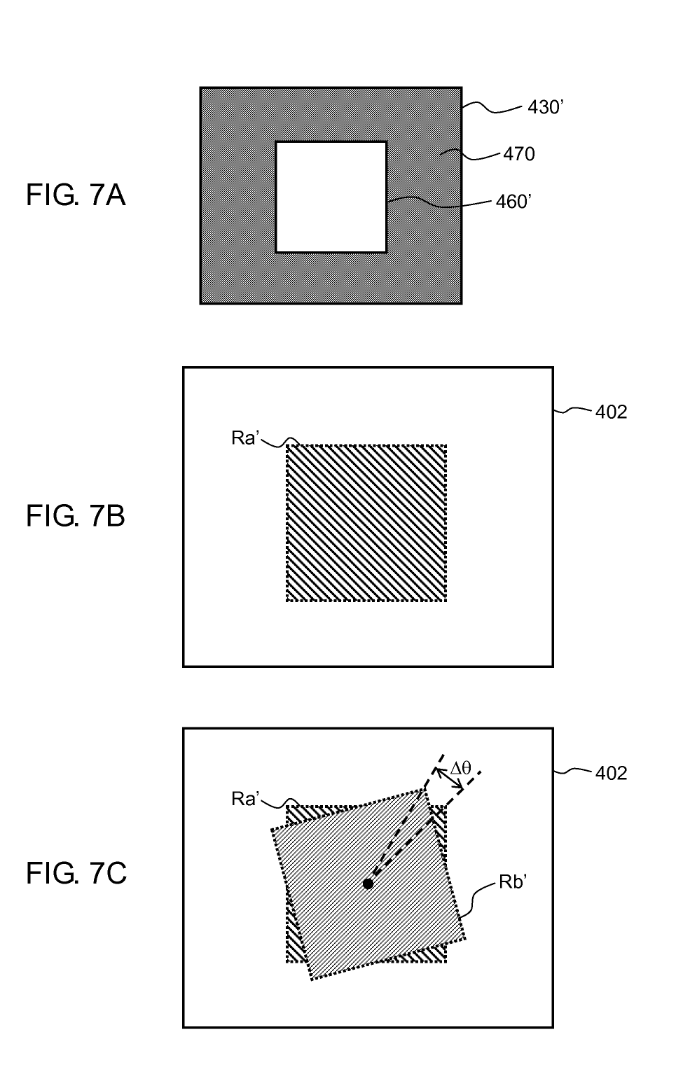

In the first exemplary embodiment, reference region image Ra is formed into the circular shape. However, there is no particular limitation to the shape of reference region image Ra. For example, reference region image Ra may have polygonal shapes such as an ellipse, a triangle, and a quadrangle or other shapes. A plurality of reference regions may be formed in one projection surface 402. The deviation adjustment method for the quadrangular reference region image will be described as an example with reference to FIGS. 7A to 7C.

FIG. 7A is a plan view of opening mask 430′. FIG. 7B illustrates the state in which the fluorescent image of white LED 410 is projected onto projection surface 402 using opening mask 430′. FIG. 7C illustrates the state in which the projection operation of surgery support system 100 as placed in FIG. 3 is performed on projection surface 402 in FIG. 7B.

As illustrated in FIG. 7A, the use of opening mask 430′ including quadrangular opening 460′ projects quadrangular reference region image Ra′ onto projection surface 402 as illustrated in FIG. 7B. In this case, whether orientations of reference region image Ra′ and projection image Rb′ differ from each other can visually be checked. For example, as illustrated in FIG. 7C, angle deviation Δθ can visually be recognized by comparing the vertices of reference region image Ra′ and projection image Rb′ to each other. Therefore, similarly to the adjustment for position deviations Δx and Δy, angle deviation Δθ can be adjusted while visually recognized.

### 3-5. Effect and the Like

As described above, in the first exemplary embodiment, deviation adjustment system 500 includes light adjustment device 400, infrared camera 210, and projector 220. Light adjustment device 400 includes projection surface 402 including reference region Ra, and emits LED light 340 including the non-visible light and the visible light from reference region Ra. Infrared camera 210 receives the non-visible light to capture the image of projection surface 402. Projector 220 projects visible-light projection image Rb onto projection surface 402 based on the image captured with infrared camera 210.

The LED light including the visible light is emitted from reference region Ra included in projection surface 402 of light adjustment device 400, and visible-light projection image Rb is projected onto projection surface 402 based on the captured image of reference region Ra. Therefore, the deviation between reference region Ra that is the object on projection surface 402 and projection image Rb is visualized, and the deviation between the object and the projection image can easily be adjusted in the projection system that captures the image of the object to project the projection image.

The deviation adjustment method is also the method for adjusting projection image G320 projected onto affected part 140 in surgery support system 100. Surgery support system 100 includes infrared camera 210 that receives infrared fluorescence 310 to capture the image of affected part 140 and projector 220 that generates visible-light projection image G320 based on the captured image of affected part 140 to project projection image G320 onto affected part 140. The deviation adjustment method includes a step of irradiating reference region Ra on projection surface 402 that is the target of the imaging and projection operations of surgery support system 100 with LED light 340 having the spectrum including the visible light component and the infrared wavelength component (including the wavelength of 850 nm). The deviation adjustment method includes a step of capturing the image of reference region Ra on projection surface 402 by using infrared camera 210. The deviation adjustment method includes a step of projecting projection image Rb based on captured reference region Ra of projection surface 402 onto projection surface 402 by using projector 220. The deviation adjustment method includes a step of comparing reference region Ra and projection image Rb to each other on projection surface 402. The deviation adjustment method includes a step of adjusting the position of projection image Rb based on the comparison result.

In the first exemplary embodiment, light adjustment device 400 adjusts projection image G320 projected onto affected part 140 in surgery support system 100. Light adjustment device 400 includes white LED 410 and projection surface 402. White LED 410 emits LED light 340 having the spectrum including the visible light component and the infrared wavelength component (including the wavelength of 850 nm). Projection surface 402 includes predetermined reference region Ra irradiated with (white) LED light 340 emitted from white LED 410, and is the target of the imaging and projection operations of surgery support system 100.

The region, in which infrared fluorescence 310 that is the fluorescence of ICG is detected, is irradiated with visible laser beam 320 in the actual surgery, and the adjustment work is performed by treating the infrared light included in white LED 410 of light adjustment device 400 as the infrared fluorescence of ICG. Therefore, the deviation between the irradiation position of visible-light laser 222 and the imaging position of infrared camera 210 is visualized on projection surface 402, the deviation can easily be adjusted. As a result, the region of affected part 140 that is detected and specified by infrared camera 210 can properly be irradiated with visible laser beam 320.

In the first exemplary embodiment, projection surface 402 is the principal surface of screen member 440, but the present disclosure is not limited thereto. Alternatively, for example, light shielding surface 470 of opening mask 430 may be used as the projection surface in the light adjustment device that does not include screen member 440. In this case, the reference region that outputs LED light 340 through opening 460 is also formed.

In the first exemplary embodiment, reference region Ra is formed by opening 460, but the present disclosure is not limited thereto. Alternatively, the reference region may be formed by guiding LED light 340 to be incident on projection surface 402 using a reflecting mirror or a lens.

In the first exemplary embodiment, the projection image is adjusted by the signal processing based on the deviation correction amount. However, the deviation adjustment method of the first exemplary embodiment is not limited to the signal processing. For example, the physical placement of infrared camera 210 or visible-light laser 222 may be adjusted while projection surface 402 of light adjustment device 400 is viewed.

In the first exemplary embodiment, the adjustment worker manipulates the manipulation unit to align reference region Ra with projection image Rb, but the present disclosure is not limited thereto. Alternatively, control device 230 may compare reference region Ra and projection image Rb to each other on projection surface 402, and adjust the position of the projection image based on the comparison result. The image of projection surface 402 is captured with a visible-light camera to specify the positions of reference region Ra and visible region Rb, and control device 230 may perform the alignment. For example, the number of dots on the image captured with the visible-light camera may be counted and converted into the correction amount. Control device 230 may perform the processing using a predetermined program.

In the first exemplary embodiment, moving amounts Δxd and Δyd are stored in memory 240 as the deviation correction amount, but the present disclosure is not limited thereto. Alternatively, correction amount Δθd of rotation angle θ and correction amount ΔZd of projection magnification Z may be stored in memory 240 in the case that rotation angle θ and projection magnification Z of projection image Rb with respect to reference region Ra are changed in the alignment of reference region Ra with projection image Rb. Projection magnification Z and correction amount ΔZd may be set in terms of zoom value in the optical system, such as a zoom lens, which projects the projection image or in terms of digital value in the signal processing of the projection image.

For example, correction amount Δθd can be extracted based on angle deviation Δθ in FIG. 7C. Correction amount ΔZd can be extracted by comparing the distance between two vertices of reference region Ra′ to the distance between two vertices of projection image Rb′ in FIG. 7C. For example, the placement of light adjustment device 400 is changed, the image of light adjustment device 400 is captured with the visible-light camera, and a distortion of the projection image may be extracted and corrected by comparing reference region Ra and projection image Rb to each other.

In the first exemplary embodiment, the deviation is adjusted using one light adjustment device 400. Alternatively, the deviation is adjusted using the plurality of light adjustment devices 400. Therefore, because the deviation is adjusted without changing the placement of light adjustment device 400, an adjustment time can be shortened, and the adjustment accuracy can be improved.

In the deviation adjustment method of the first exemplary embodiment, projector 220 includes visible-light laser 222, and the scan is performed with the laser irradiation. The method for projecting the projection image is not limited to the first exemplary embodiment, but the deviation adjustment method can be performed with the light adjustment device 400 in the case that the projection image is projected by another system.

### 4. Scan Operation by Laser Scan Type Projection

### 4-1. Outline of Scan Operation

Sometimes a lighting device having high illuminance (30000 lux to 100000 lux), such as shadowless lamp 120 and a lighting fixture mounted on a doctor's head, is used in the surgery performed with surgery support system 100. The light source used in a usual imaging and irradiation device 200 has illuminance as low as several hundreds lux, and the projection image becomes inconspicuous to be hardly visually recognized under a high-illuminance environment.

The laser scan type projection in which projector 220 including visible-light laser 222 and MEMS mirror 221 is used is adopted in surgery support system 100 of the present disclosure. Specifically, while the high-illuminance light can be supplied from visible-light laser 222, only the inside or boundary of the region of affected part 140, which is detected and specified by infrared camera 210, is scanned with visible laser beam 320 using MEMS mirror 221. Therefore, in consideration of the safety, the projection image can easily visually be recognized even in the high-illuminance environment.

### 4-2. Detailed Scan Operation

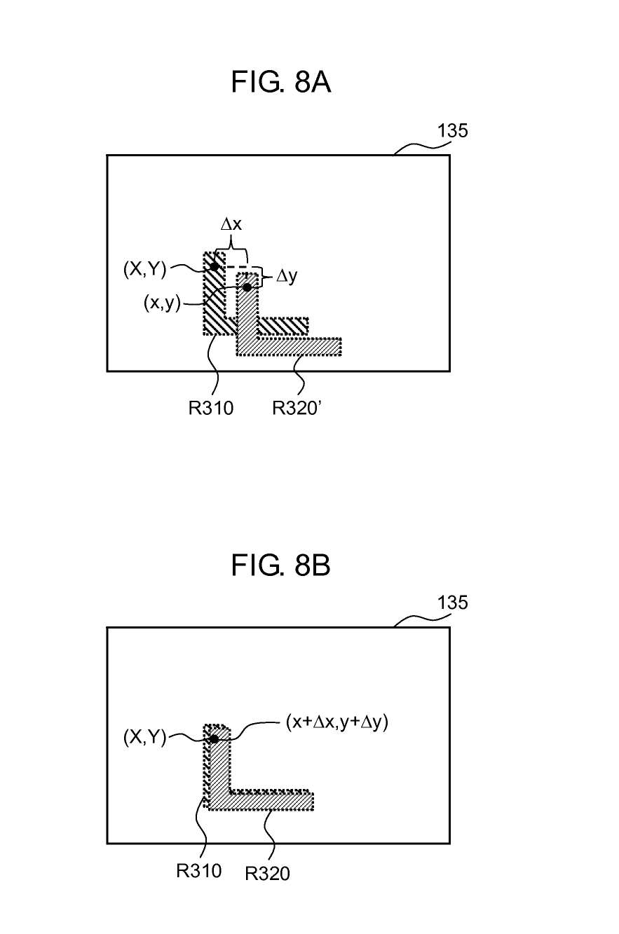

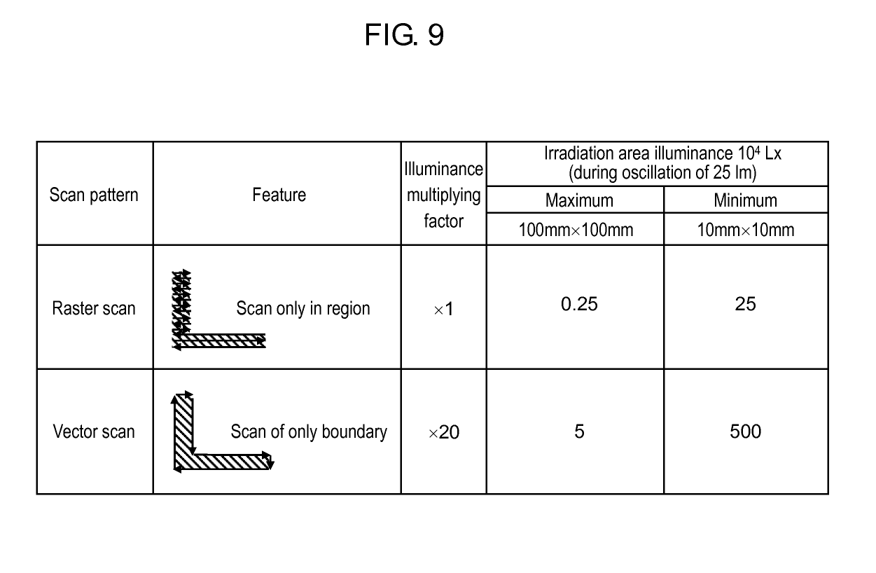

A scan operation performed with visible-light laser 222 and MEMS mirror 221 will be described below with reference to FIGS. 1, 8A, 8B, and 9. FIGS. 8A and 8B are views illustrating infrared fluorescence 310 and visible laser beam 320 before and after the deviation is adjusted. FIG. 9 is a view illustrating a scan pattern of visible-light laser 222 and MEMS mirror 221.

As illustrated in FIG. 1, surgical table 110 on which patient 130 is placed is disposed at the position facing the imaging surface of infrared camera 210 and the irradiation port of visible laser beam 320, the position being located directly below imaging and irradiation device 200. At this point, assuming that the acceptable range is set, for example, to 1000 mm±300 mm based on the focal distance of infrared camera 210, a use height of imaging and irradiation device 200 or surgical table 110 is adjusted such that the body axis of patient 130 is located at the position where the distance from the bottom surface of imaging and irradiation device 200 becomes 1000 mm.

It is assumed that ICG is already injected into the blood of patient 130, and that ICG is accumulated in affected part 140. The operation of surgery support system 100 is started in the state in which patient 130 is placed on surgical table 110 while a body portion to which a knife is put is facing upward with respect to affected part 140.

Control device 230 controls infrared excitation light source 250 such that surgical field 135 near affected part 140 of patient 130 is irradiated with infrared excitation light 300 around the ICG excitation wavelength of 800 nm. ICG accumulated in affected part 140 generates an excitation reaction by infrared excitation light 300, and emits infrared fluorescence 310 having a peak wavelength of about 850 nm. Infrared fluorescence 310 emitted from ICG accumulated in affected part 140 is partially transmitted through dichroic mirror 211. Infrared camera 210 receives infrared fluorescence 310 transmitted through dichroic mirror 211, and captures the image of surgical field 135. For this reason, infrared fluorescence region R310 emitting infrared fluorescence 310 is also taken in the image captured by infrared camera 210. Infrared camera 210 sends the captured image to control device 230.

Control device 230 specifies the coordinate (for example, the XY-coordinate from one vertex of the captured image) in the emission region of infrared fluorescence 310 based on the captured image sent from infrared camera 210. At this point, control device 230 reads position deviations Δx and Δy that are the deviation correction amounts stored in memory 240. Control device 230 calculates a correction coordinate in which the coordinate specified based on the captured image sent from infrared camera 210 is corrected by the deviation correction amounts read from memory 240. Control device 230 controls MEMS mirror 221 such that the scan coordinate corresponding to the correction coordinate of the coordinate in the captured image sent from infrared camera 210 is irradiated with visible laser beam 320 using a predetermined laser scan pattern. The laser scan pattern is described in detail later.

FIG. 8A illustrates infrared fluorescence region R310 of infrared fluorescence 310 of ICG and projection region R320′ in visible laser beam 320 in the case that the correction is not performed based on the deviation correction amount. In the case that the correction coordinate corrected by the deviation correction amount is not used, the position deviating from infrared fluorescence region R310 of ICG by position deviations Δx and Δy is irradiated with visible laser beam 320.

On the other hand, FIG. 8B illustrates infrared fluorescence region R310 of infrared fluorescence 310 of ICG and projection region R320 in visible laser beam 320 in the case that the correction is performed based on the deviation correction amount. In the case that the correction coordinate corrected by the deviation correction amount is used, infrared fluorescence region R310 of ICG is accurately irradiated with visible laser beam 320.

As described above, when the correction coordinate is used, the region of affected part 140 emitting infrared fluorescence 310 can accurately be irradiated with visible laser beam 320.

The laser scan pattern of visible-light laser 222 and MEMS mirror 221 will be described below. FIG. 9 illustrates a raster scan and a vector scan, which can be selected as the laser scan pattern in surgery support system 100.

The raster scan is a scan pattern, in which the operation to reciprocally irradiate the region of affected part 140 emitting infrared fluorescence 310 with visible laser beam 320 is performed only on the inside surface of the region such that the surface is painted with visible laser beam 320. The illuminance multiplying rate is set to one in the raster scan of FIG. 9. During the oscillation with 25 lumens, irradiation surface illuminance becomes about 2500 lux when an irradiation area is maximized (100 mm by 100 mm), and the irradiation surface illuminance becomes about 250000 lux when the irradiation area is minimized (10 mm by 10 mm).

The vector scan is a scan pattern, in which the operation to irradiate the region of affected part 140 emitting infrared fluorescence 310 with visible laser beam 320 is performed only on the boundary of the region such that a line is drawn with visible laser beam 320. The illuminance multiplying rate is set to 20 in the vector scan of FIG. 9. During the oscillation with 25 lumens, the irradiation surface illuminance becomes about 50000 lux when the irradiation area is maximized (100 mm by 100 mm), and the irradiation surface illuminance becomes about 5000000 lux when the irradiation area is minimized (10 mm by 10 mm).

The doctor can switch between the visible-light laser irradiation with the raster scan and the visible-light laser irradiation with the vector scan by manipulating the manipulation unit (not illustrated) according to a surgical content.

In FIG. 9, the raster scan and the vector scan are illustrated as the scan pattern, but the present disclosure is not limited thereto. Alternatively, for example, a pattern in which the scan is properly interleaved while only the inside of the region of affected part 140 emitting infrared fluorescence 310 is scanned may be used as a derivative pattern of the raster scan. Alternatively, a pattern in which the irradiation position is shifted to another region after the region is continuously scanned a plurality of times may be used as a derivative pattern of the raster scan or the vector scan.

Control device 230 causes projector 220 to irradiate the region of affected part 140 emitting infrared fluorescence 310 with visible laser beam 320 to project the projection image based on the set scan pattern. At this point, control device 230 controls MEMS mirror 221 such that the region of affected part 140 is irradiated with the visible-light laser based on the set scan pattern. Control device 230 continuously performs the scan operation after the surface or boundary of the region of affected part 140 emitting infrared fluorescence 310 is scanned.

### 4-3. Effect and the Like

As described above, in the first exemplary embodiment, surgery support system 100 includes infrared camera 210, projector 220, and control device 230. Infrared camera 210 captures an image of affected part 140. Based on the image captured with infrared camera 210, projector 220 generates the visible-light projection image G320, and projects projection image G320 onto affected part 140. Control device 230 controls the operations of infrared camera 210 and projector 220. Projector 220 includes visible-light laser 222 that emits visible laser beam 320. Control device 230 controls projector 220 such that projection region R320 onto which projection image G320 is projected is scanned with visible laser beam 320 using the predetermined scan pattern.

In surgery support system 100, the high-illuminance laser source is used as the irradiation light source, so that visibility can be enhanced even in the high-illuminance environment caused by another lighting device such as shadowless lamp 120. Additionally, because only the inside or boundary of the specific region is scanned using the predetermined scan pattern, the illuminance can be obtained to enhance the visibility compared with the case that the wide region is irradiated. The same place is not continuously irradiated with the high-illuminance visible laser beam 320, but the irradiation position is scanned. Therefore, surgery support system 100 can be provided that facilitates the visual recognition of the projection image in the high-illuminance environment even in consideration of the safety.

The scan pattern may be the raster scan in which the inside of projection region R320 is scanned with visible laser beam 320. The scan pattern may be the vector scan in which the boundary of projection region R320 is scanned with visible laser beam 320.

Projector 220 may further include MEMS mirror 221 that includes the plurality of micro-mirror surfaces reflecting visible laser beam 320. Control device 230 may control projector 220 such that the tilt angle of each micro-mirror surface of MEMS mirror 221 is changed to scan projection region R320 with visible laser beam 320. Therefore, processing amount can be decreased in the scan of visible laser beam 320.

### 5. Cutting Auxiliary Line Projecting Operation According to Detection of Affected Part

### 5-1. Outline of Cutting Auxiliary Line Projecting Operation

In starting the surgery of affected part 140, it is necessary for the doctor to determine a cutting position to which the knife is put. Therefore, the doctor performs the work to check a relationship between affected part 140 and the cutting position to which the knife is put with an image analysis device. At this point, the doctor plans the cutting position so as to put the knife to the cutting position with a margin of a given distance with respect to affected part 140. The doctor memorizes the planned cutting position and performs the surgery.

However, it is not easy to correctly reproduce the cutting position that is planned before the start of the surgery, and a burden is applied to the doctor. In addition, this also causes time to be wasted in starting the surgery.

Therefore, the inventor devises the projection operation in which, in addition to the projection of visible-light projection image G320 displaying the region of affected part 140 in which ICG is accumulated, cutting auxiliary line 321 is projected in order to support the determination of the cutting position to which the knife is put. Therefore, the reproduction of the cutting position that is planned before the start of the surgery can be supported to reduce the burden on the doctor. Additionally, time wasted in starting the surgery can be shortened.

### 5-2. Detailed Cutting Auxiliary Line Projecting Operation

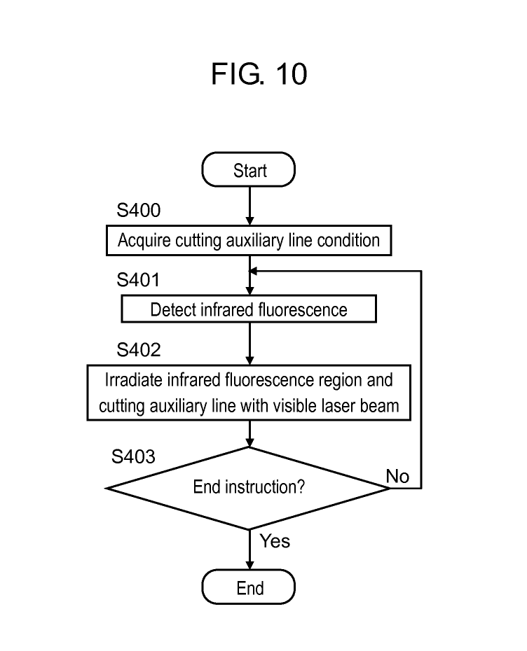

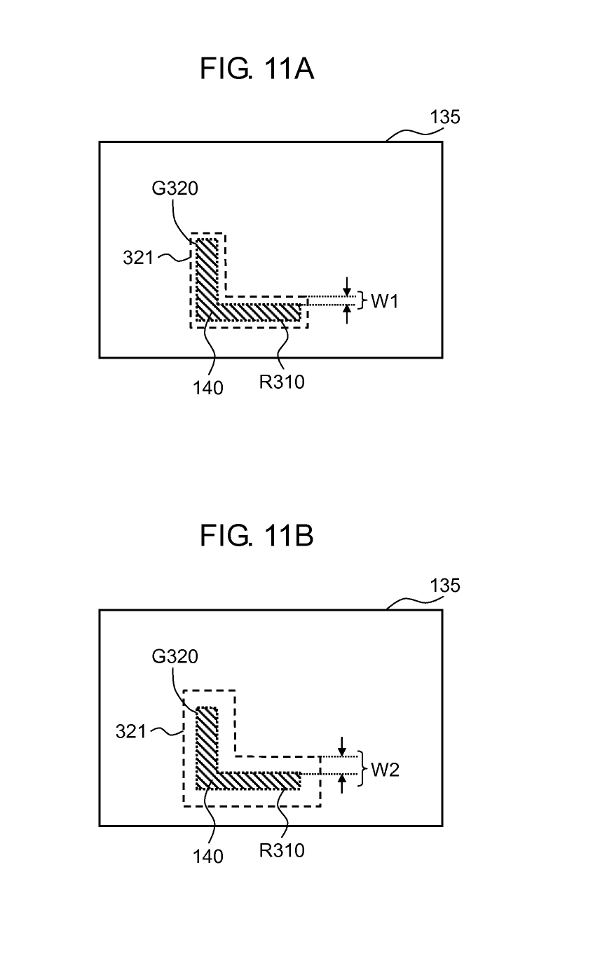

An operation to project cutting auxiliary line 321 according to the detection of affected part 140 will be described below with reference to FIGS. 10, 11A, and 11B. FIG. 10 is a flowchart illustrating the operation to project cutting auxiliary line 321 according to the detection of affected part 140. FIGS. 11A and 11B are views illustrating the operation to project cutting auxiliary line 321 according to the detection of affected part 140.

It is assumed that a doctor plans the cutting position so as to put the knife to the cutting position with a margin of a given distance (hereinafter, referred to as a “cutting margin width”) with respect to affected part 140 in advance of the start of the surgery supported with surgery support system 100. It is also assumed that the doctor inputs the planned cutting margin width to surgery support system 100 using the manipulation unit (not illustrated). For example, for the planned cutting margin width of 2 cm, the doctor inputs information indicating the cutting auxiliary line condition of the cutting margin width of 2 cm to surgery support system 100. Control device 230 of surgery support system 100 stores the cutting margin width in memory 240 based on the input information.

The flow in FIG. 10 is started, when the surgery supported with surgery support system 100 is started with the information indicating the cutting auxiliary line condition stored in memory 240.

Control device 230 reads the cutting margin width stored in memory 240, and acquires the cutting auxiliary line condition (S400).

Then, control device 230 causes infrared camera 210 to capture the fluorescent image of infrared fluorescence 310 that is emitted from ICG by the reaction with infrared excitation light 300 (S401). At this point, control device 230 specifies the coordinate of the region emitting the infrared fluorescence from the captured image sent from infrared camera 210. Control device 230 further reads the deviation correction amount from memory 240, and calculates the correction coordinate in which the coordinate specified based on the captured image sent from infrared camera 210 is corrected by the deviation correction amounts. Thus, control device 230 detects infrared fluorescence region R310 of affected part 140.

Then, control device 230 starts the irradiation of the infrared fluorescent region and cutting auxiliary line 321 with visible laser beam 320 based on the calculated correction coordinate (S402). At this point, control device 230 calculates the position to which cutting auxiliary line 321 is projected based on detected infrared fluorescence region R310 and the cutting margin width acquired in step S400. Then, control device 230 controls MEMS mirror 221 such that cutting auxiliary line 321 is projected to the position distant from the region specified as affected part 140 by the cutting margin width while the laser scan is performed on the region specified as affected part 140.

In the processing of step S402, control device 230 adjusts the projection magnification based on the distance information detected by the TOF sensor 260. In the case that the cutting margin width is set to 2 cm, control device 230 controls MEMS mirror 221 such that cutting auxiliary line 321 is projected to the position distant from the region specified as affected part 140 by 2 cm. Therefore, in the surrounding of the region specified as affected part 140, cutting auxiliary line 321 is projected to the position 2 centimeter away so as to be similar to the region specified as affected part 140. The projection of cutting auxiliary line 321 will be described in detail with reference to FIGS. 11A and 11B.

FIG. 11A illustrates surgical field 135 in the state in which the operation to project cutting auxiliary line 321 is performed according to the detection of affected part 140 in the case that first cutting margin width W1 is set. FIG. 11B illustrates surgical field 135 in the state in which the operation to project cutting auxiliary line 321 is performed according to the detection of affected part 140 in the case that second cutting margin width W2 is set. It is assumed that second cutting margin width W2 is set larger than first cutting margin width W1.

Referring to FIGS. 11A and 11B, visible-light projection image G320 is projected onto infrared fluorescence region R310 of affected part 140 emitting infrared fluorescence 310 in surgical field 135 by the detection of infrared fluorescence 310 in the captured image. Based on the distance information detected by TOF sensor 260 in addition to the irradiation position of projection image G320, control device 230 sets the irradiation position with visible laser beam 320 projecting cutting auxiliary line 321 such that infrared fluorescence region R310 is surrounded with gaps of cutting margin widths W1 and W2 in surgical field 135. Therefore, as illustrated in FIGS. 11A and 11B, surgery support system 100 can change the position to which cutting auxiliary line 321 is projected according to the cutting position plan (cutting margin width) of the doctor.

Returning to FIG. 10, control device 230 repeats the pieces of processing in S401 and S402 until the doctor and the like issue an end instruction using the manipulation (NO in S403). When the end instruction is issued (YES in S403), control device 230 ends the irradiation operation with visible laser beam 320.

In the description of the flow in FIG. 10, the condition of cutting auxiliary line 321 is cutting margin widths W1 and W2. The condition of cutting auxiliary line 321 is not limited to cutting margin widths W1 and W2. For example, a threshold in an intensity distribution of infrared fluorescence 310 may be used as the condition of cutting auxiliary line 321. In this case, in the processing of step S402, control device 230 controls projector 220 such that the boundary of the intensity distribution in the captured image is extracted based on the image captured with infrared camera 210 and the threshold set as the condition of cutting auxiliary line 321, and such that cutting auxiliary line 321 is projected onto the extracted boundary. For example, in enucleating a part of an organ (for example, one zone in a Couinaud classification of a lever), in the case that the blood flow or the like is restricted to inject ICG such that the portion to be enucleated emits the fluorescence, the doctor and the like can visually recognize the cutting position of the portion to be enucleated on the surface of the organ.

Cutting auxiliary line 321 may be projected to the position distant from the boundary of the intensity distribution in the captured image by cutting margin widths W1 and W2 using the threshold in the intensity distribution of the infrared fluorescence and cutting margin widths W1 and W2 as the condition of cutting auxiliary line 321. In the case that cutting auxiliary line 321 is projected onto the boundary of the intensity distribution of the infrared fluorescence, control device 230 may fix the irradiation position based on an image analysis of the image captured with infrared camera 210 with no use of the distance information from TOF sensor 260.

### 5-3. Effect and the Like

As described above, in the first exemplary embodiment, surgery support system 100 includes infrared camera 210, projector 220, and control device 230. Infrared camera 210 captures an image of affected part 140. Projector 220 generates visible-light projection image G320, and projects projection image G320 onto affected part 140. Control device 230 detects infrared fluorescence region R310 of affected part 140 emitting infrared fluorescence 310 based on the image captured with infrared camera 210. Control device 230 controls projector 220 such that projection image G320 indicating detected infrared fluorescence region R310 is projected, and such that cutting auxiliary line 321 that is the projection image indicating the auxiliary line at the position corresponding to a predetermined condition is projected onto detected infrared fluorescence region R310.

Therefore, based on the cutting margin width input by the doctor in advance of the start of the surgery, cutting auxiliary line 321 can be irradiated in addition to the irradiation of the region specified as affected part 140. Therefore, the reproduction of the cutting position that is planned before the start of the surgery can be supported to reduce the burden on the doctor. Additionally, the time wasted in starting the surgery can be shortened.

In surgery support system 100, cutting auxiliary line 321 is projected based on infrared fluorescence region R310 of affected part 140 that is detected based on the emission of infrared fluorescence 310. Therefore, the doctor and the like can visually recognize the auxiliary line that is matched with the position of affected part 140 in real time in surgical field 135.

In surgery support system 100, the position to which cutting auxiliary line 321 is projected may be set to the boundary of the intensity distribution based on the intensity distribution of the infrared fluorescence in the captured image.

In surgery support system 100, the predetermined condition may be cutting margin widths W1 and W2 indicating the distance from detected infrared fluorescence region R310.

Surgery support system 100 may further include TOF sensor 260 that detects distance information indicating the distance to affected part 140. Based on the distance information detected by TOF sensor 260, control device 230 may project cutting auxiliary line 321 to the position distant from detected infrared fluorescence region R310 by cutting margin widths W1 and W2.

In the first exemplary embodiment, in the case that the cutting margin width that is the predetermined condition is set to 2 cm, cutting auxiliary line 321 is uniformly projected to the position distant from the region specified as affected part 140 by 2 cm, but the present disclosure is not limited thereto. Alternatively, the position to which cutting auxiliary line 321 should be projected with respect to the region specified as affected part 140 may be changed according to the position of the cutting margin width.

In the first exemplary embodiment, the projection of cutting auxiliary line 321 may properly be turned on and off in response to the manipulation of the doctor and the like while the region specified as affected part 140 is irradiated with visible laser beam 320. In the case that the projection of cutting auxiliary line 321 is turned off, cutting auxiliary line 321 is not projected, but only the region specified as affected part 140 is irradiated with visible laser beam 320.

In the first exemplary embodiment, the condition of cutting auxiliary line 321 (cutting margin width) is input in advance of the start of the surgery, but the present disclosure is not limited thereto. Alternatively, the condition of cutting auxiliary line 321 may be changed in response to the manipulation of the doctor and the like during the surgery.

### 6. Operation to Project Surgical Auxiliary Information to Surrounding of Affected Part

### 6-1. Outline of Surgical Auxiliary Information Projecting Operation

The doctor performs the surgery while properly checking vital data of patient 130. Examples of the vital data include a blood pressure, the number of heart beats (number of pulses), an oxygen concentration, and an electrocardiogram. The doctor can perform the surgery according to a change in condition of patient 130 by checking the vital data. The doctor performs the surgery while properly checking an inspection image of patient 130. Examples of the inspection image include an MRI (Magnetic Resonance Imaging) image, a CT (Computed Tomography) image, and an X-ray image. The doctor can perform the surgery according to an inspection result of patient 130 by checking the inspection image. As needed basis, the doctor performs the surgery while checking a memorandum in which a surgical procedure and precautions in the surgery are described.

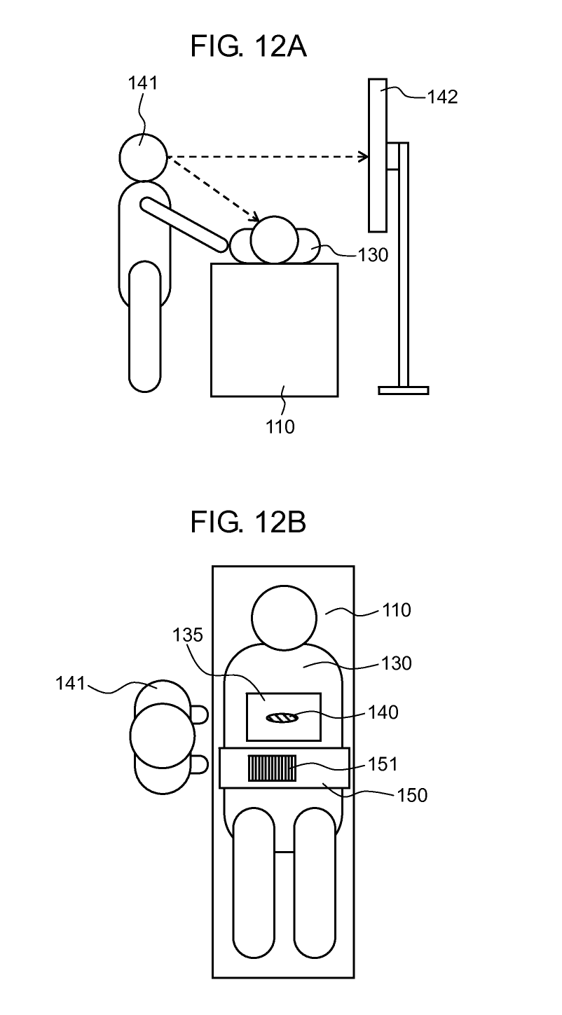

Thus, the doctor performs the surgery while properly checking surgical auxiliary information such as the vital data, the inspection image, and the surgical procedure. FIG. 12A illustrates a state of a conventional surgery. The surgical auxiliary information is displayed on monitor 142. Doctor 141 performs the surgery on patient 130 while checking the surgical auxiliary information displayed on monitor 142. Because doctor 141 performs the surgery while moving a visual line between monitor 142 and patient 130, the burden is applied to doctor 141, and doctor 141 spends the checking time.

The inventor devises the projection operation in which, in addition to the projection of the visible-light image projected onto the region specified as affected part 140, surgical auxiliary information 151 is projected to the surrounding of affected part 140. Therefore, the doctor and the like can reduce the movement of the visual line during the surgery. As a result, the burden on the doctor and the like can be reduced to shorten the checking time.

### 6-2. Detailed Surgical Auxiliary Information Projecting Operation

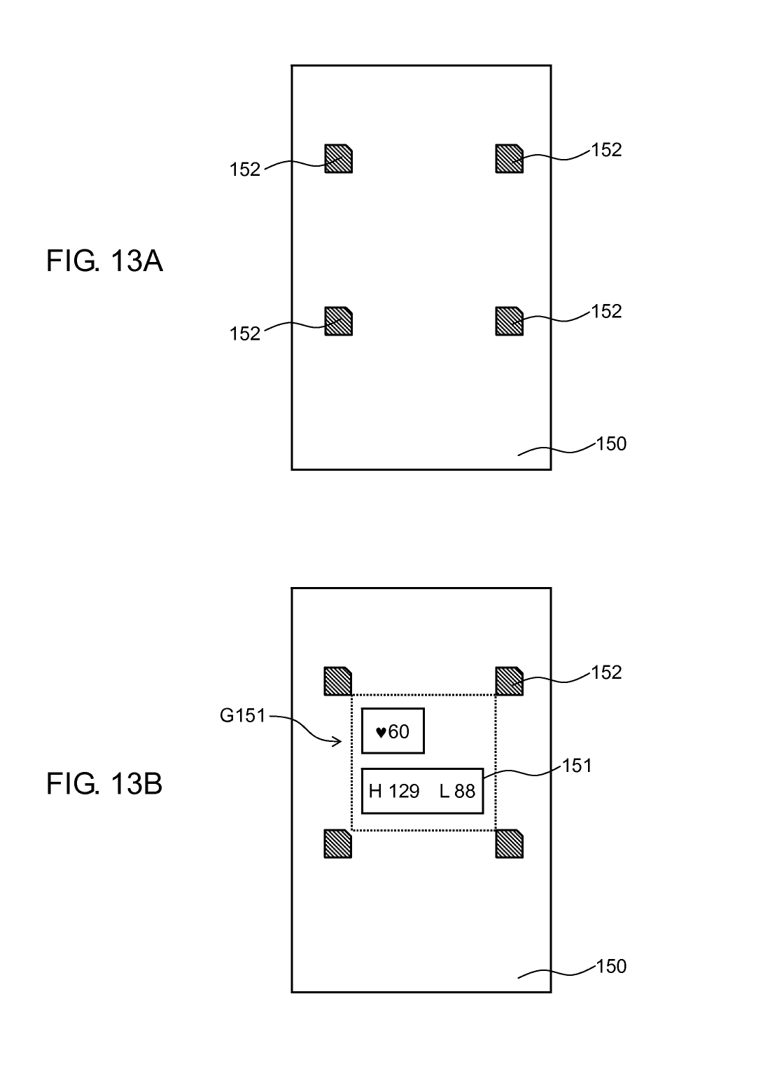

The projection of the surgical auxiliary information to a surrounding of affected part 140 will be described below with reference to FIGS. 12B, 13A, and 13B. FIG. 12B is a view illustrating the projection of surgical auxiliary information 151 to the surrounding of affected part 140. FIGS. 13A and 13B are views illustrating the projection of surgical auxiliary information 151 onto auxiliary screen member 150.

Control device 230 of surgery support system 100 is communicably connected to a medical instrument (not illustrated) that acquires various pieces of vital data. Therefore, control device 230 acquires the vital data necessary for the surgery in real time from the communicably-connected medical instrument.

The inspection image data of patient 130 and the memorandum such as the surgical procedure are previously stored in memory 240 in advance of the start of the surgery by the manipulation unit manipulated by the doctor 141. Therefore, control device 230 reads and acquires the inspection image data necessary for the surgery and the memorandum such as the surgical procedure from memory 240.

FIG. 12B illustrates the state of the projection of surgical auxiliary information 151 in the first exemplary embodiment. In starting the surgery, as illustrated in FIG. 12B, doctor 141 disposes auxiliary screen member 150 projecting surgical auxiliary information 151 in the neighborhood of affected part 140 of patient 130. Any material may be used for auxiliary screen member 150 as long as the projection image can be displayed on the material. Auxiliary screen member 150 may have any shape and any size as long as auxiliary screen member 150 can be disposed near affected part 140. In the example of FIG. 12B, auxiliary screen member 150 is disposed on the right of affected part 140 when viewed from doctor 141. However, there is no limitation to the placement position of auxiliary screen member 150. Auxiliary screen member 150 may be disposed in any place around affected part 140 according to a dominant arm of the doctor or the like who uses surgery support system 100, checking easiness, or a surgery content.

FIG. 13A is a top view illustrating auxiliary screen member 150 in the state in which the surgical auxiliary information is not projected. As illustrated in FIG. 13A, marker 152 is added to an upper surface of auxiliary screen member 150. Marker 152 is positioned on auxiliary screen member 150 as a reference indicating the region where surgical auxiliary information 151 is displayed on auxiliary screen member 150.

A camera (not illustrated) is connected to control device 230 of surgery support system 100, and the camera captures the image of marker 152 added onto auxiliary screen member 150. The camera sends the captured image of marker 152 to control device 230. A correspondence relationship between the coordinates of the imaging region in the image captured with the camera and the projection region of surgical auxiliary information in visible-light laser 222 is previously stored in memory 240. Control device 230 specifies the region onto which surgical auxiliary information 151 is projected from the correspondence relationship stored in memory 240 and the detection result of the position of marker 152 from the sent captured image. Control device 230 controls MEMS mirror 221 such that surgical auxiliary information 151 is projected onto the specified region. Therefore, as illustrated in FIG. 13B, projection image G151 indicating surgical auxiliary information 151 is projected onto the upper surface of auxiliary screen member 150.

Surgery support system 100 projects projection image G151 of surgical auxiliary information 151 onto auxiliary screen member 150 while projecting projection image G320 onto infrared fluorescence region R310 specified as affected part 140 (refer to FIG. 2B). Therefore, the doctor can reduce the movement of the visual line during the surgery. As a result, the burden on doctor 141 can be reduced to shorten the checking time, and the surgery can be supported.

In the first exemplary embodiment, surgical auxiliary information 151 is projected onto auxiliary screen member 150, but the present disclosure is not limited thereto. Alternatively, surgical auxiliary information 151 may not be projected onto auxiliary screen member 150 but may directly be projected onto a body surface of patient 130. At this point, marker 152 may be provided to the body surface of patient 130.

In the first exemplary embodiment, the image of marker 152 is captured with the camera, but the present disclosure is not limited thereto. Alternatively, for example, the image of marker 152 may be captured with infrared camera 210. In this case, for example, marker 152 is formed with a material into which ICG is applied, kneaded, or flown. Therefore, the images of affected part 140 and marker 152 can be captured only with infrared camera 210.

In the first exemplary embodiment, the region onto which surgical auxiliary information 151 is projected is specified by marker 152, but the present disclosure is not limited thereto. Alternatively, the region onto which surgical auxiliary information 151 is projected may be specified with no use of marker 152. For example, surgical auxiliary information 151 may be projected to the position distant from the position where affected part 140 is irradiated with visible laser beam 320 by a distance in a direction previously set by the doctor.

For example, it is assumed that surgical auxiliary information 151 is previously set so as to be projected to the position distant from a right end of the region specified as affected part 140 by 20 cm in the right side when viewed from doctor 141. At this point, control device 230 controls MEMS mirror 221 such that surgical auxiliary information 151 is projected to the position that is previously set with respect to the region specified as affected part 140. Therefore, surgical auxiliary information 151 can be projected to any place that is easily checked by doctor 141. Control device 230 may calculate the position to which surgical auxiliary information 151 is projected in surgical field 135 based on the distance information detected by TOF sensor 260.

### 6-3. Effect and the Like

As described above, in the first exemplary embodiment, surgery support system 100 includes infrared camera 210, projector 220, and control device 230. Infrared camera 210 captures an image of affected part 140. Projector 220 generates visible-light projection image G320, and projects projection image G320 onto affected part 140. Control device 230 controls the projection operation of projector 220 based on the image captured with infrared camera 210. Control device 230 controls projector 220 such that projection image G320 indicating the captured image of affected part 140 is projected, and such that projection image G151 indicating surgical auxiliary information 151 that is the information on the surgery of affected part 140 is projected to the neighborhood of affected part 140.

Therefore, projection image G151 is projected to the neighborhood of affected part 140, the movement of the visual line from affected part 140 is reduced when the doctor and the like check surgical auxiliary information 151, and the burden on the doctor and the like can be reduced during the surgery.

Surgery support system 100 may further include auxiliary screen member 150, which is disposed near affected part 140 and includes marker 152. In this case, control device 230 projects projection image G151 onto auxiliary screen member 150 based on the position of marker 152. Surgery support system 100 may further include the camera that captures the image of marker 152, or the image of marker 152 may be captured with infrared camera 210.

Surgery support system 100 may further include memory 240 in which surgical auxiliary information 151 is stored. Control device 230 may acquire surgical auxiliary information 151 through the communication with an external device.

Surgery support system 100 may further include a distance detector such as TOF sensor 260 that detects distance information indicating the distance to affected part 140. Control device 230 may cause projector 220 to project surgical auxiliary information 151 to the position distant from affected part 140 by the predetermined distance based on the detected distance information. Control device 230 may cause projector 220 to project surgical auxiliary information 151 onto the substantially flat region near affected part 140 based on the detected distance information. For example, the distance detector may output a distance image as the distance information.

### 7. Monitoring of Use Height of Imaging and Irradiation Device

### 7-1. Outline of Use Height Monitoring Operation

In surgery support system 100 of FIG. 1, at the beginning of the surgery, the height acceptable range is set to 1000 mm±300 mm based on the focal distance of infrared camera 210, and the use heights of imaging and irradiation device 200 and surgical table 110 are adjusted such that the body axis of patient 130 is located at the position where the distance from the bottom surface of imaging and irradiation device 200 becomes 1000 mm. During the surgery, the orientation of patient 130 is changed according to the surgery content, or the placement of imaging and irradiation device 200 is changed in association with a turnover of a practitioner. Therefore, the use heights of imaging and irradiation device 200 and surgical table 110 are changed.

In the present disclosure, the distance detector is provided in imaging and irradiation device 200 to monitor the use height of imaging and irradiation device 200 during the surgery. Therefore, the orientation of patient 130 can be changed or the height of the surgical table can be adjusted according to the surgery content within the use height acceptable range. On the other hand, when the use height is outside the acceptable range, a warning is issued to be able to avoid false recognition of a user such as the doctor. When the use height is outside the acceptable range, the control is performed such that the projection image is not projected, which allows the security assurance during the surgery.

In the first exemplary embodiment, TOF sensor 260 that emits infrared detection light 330 having wavelengths of 850 nm to 950 nm is used as the distance detector. Infrared detection light 330 emitted from TOF sensor 260 is reflected by the body surface of patient 130, and returned to and received by TOF sensor 260. At this point, infrared detection light 330 reflected by the body surface of patient 130 reaches not only TOF sensor 260 but also infrared camera 210.

In the configuration of the first exemplary embodiment, contradictory control is performed on TOF sensor 260 and infrared excitation light source 250 or visible-light laser 222 in order that the safe use height is monitored while the surgery support is performed by the detection of infrared fluorescence 310.

### 7-2. Detailed Use Height Monitoring Operation

The detailed use height monitoring operation will be described below.

### 7-2-1. Processing Flow

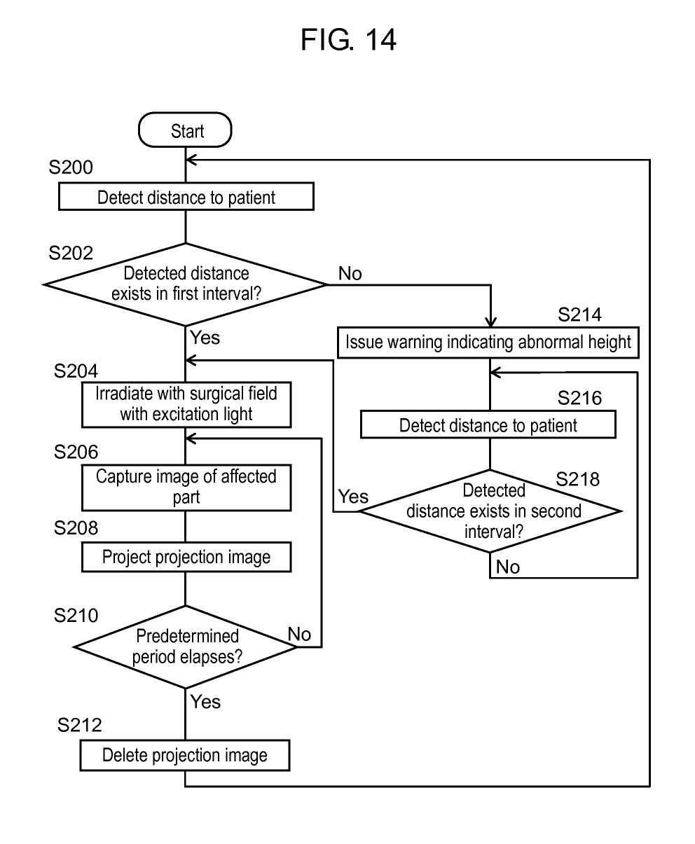

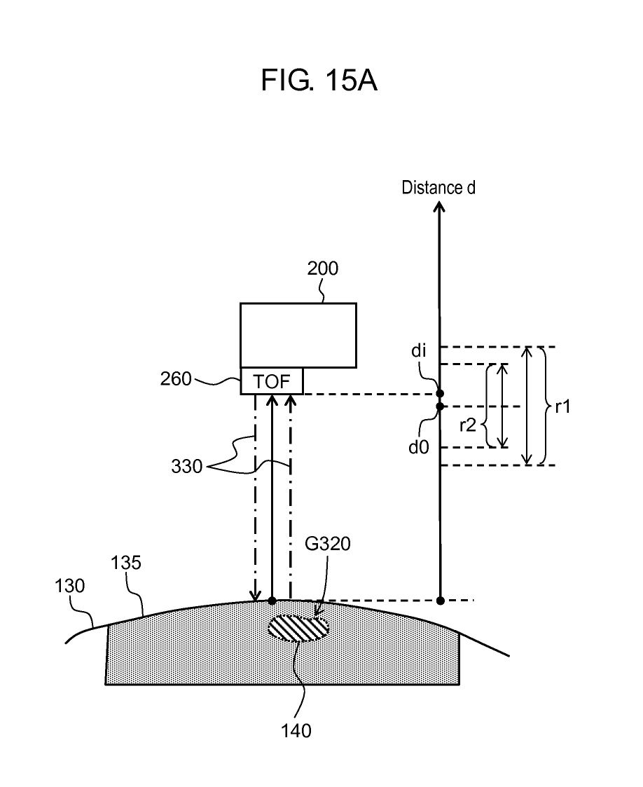

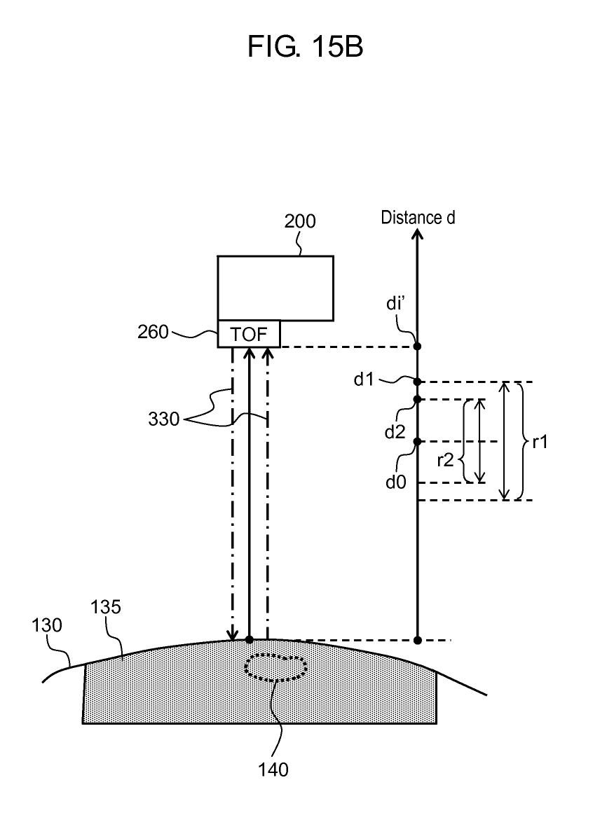

A processing flow in a use height monitoring operation of surgery support system 100 will be described with reference to FIGS. 14, 15A, and 15B. FIG. 14 is a flowchart illustrating the processing flow in the use height monitoring operation. FIGS. 15A and 15B are views illustrating the use height monitoring operation. Control device 230 of surgery support system 100 performs the flow (refer to FIG. 1).

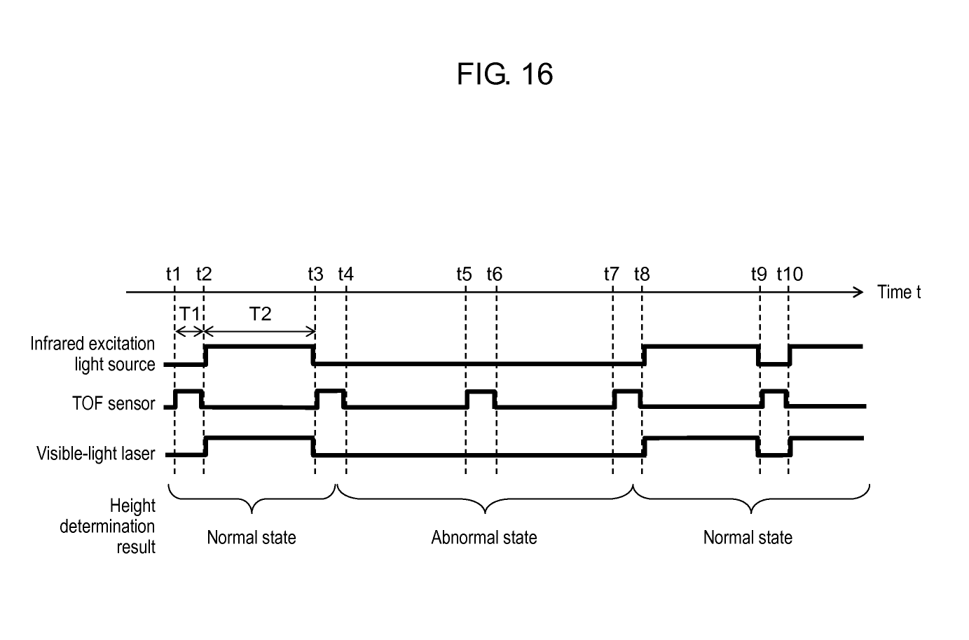

In the flow of FIG. 14, under the control of control device 230, TOF sensor 260 emits infrared detection light 330, and receives the reflected light of infrared detection light 330 to detect distance di to patient 130 as illustrated in FIG. 15A (S200). In step S200, TOF sensor 260 emits infrared detection light 330 only for a predetermined period T1 (refer to FIG. 16). TOF sensor 260 outputs the detected distance information to control device 230.

Based on the distance information from TOF sensor 260, control device 230 determines whether detected distance di falls within a range of predetermined first interval r1 (S202). First interval r1 indicates the acceptable range where surgery support system 100 can normally be operated in the distance between imaging and irradiation device 200 and affected part 140. In the first exemplary embodiment, d0 of 1000 mm is used as the standard distance, and first interval r1 is set to 1000±300 mm.

As illustrated in FIG. 15B, when di′ is outside first interval r1 while detected as distance di (NO in S202), control device 230 issues the warning indicating the abnormal use height (S214). As to the warning in step S214, for example, a message or a warning sound indicating that the use height is in an “abnormal state” is generated from a speaker (not illustrated). In step S214, the projection image is not projected onto affected part 140 as illustrated in FIG. 15B.

After period T1 elapses, control device 230 controls TOF sensor 260 to detect distance di to patient 130 similarly to the processing in step S200 (S216).

Then, control device 230 determines whether detected distance di falls within a range of predetermined second interval r2 (S218). Second interval r2 indicates that surgery support system 100 is located at the position where surgery support system 100 can be returned from the abnormal state. Second interval r2 is shorter than first interval r1. For example, second interval r2 is set to 1000±200 mm.

When it is determined that detected distance di is outside second interval r2 (NO in S218), control device 230 repeats the processing in step S216 with a predetermined period. On the other hand, when it is determined that detected distance di falls within second interval r2 (YES in S218), control device 230 sequentially performs the pieces of processing from step S204.

When it is determined that detected distance di falls within first interval r1 as illustrated in FIG. 15A (YES in S202), control device 230 controls infrared excitation light source 250 (refer to FIG. 1) to irradiate surgical field 135 with infrared excitation light 300 (S204).

During the irradiation of surgical field 135 with infrared excitation light 300, control device 230 controls infrared camera 210 to capture the image of affected part 140 of surgical field 135 (S206). Based on the captured image in the processing in step S206, control device 230 causes projector 220 to project the visible-light projection image G320 (S208). The pieces of processing in steps S202, 204, and 206 are performed similarly to the basic projection operation in surgery support system 100 described above (refer to FIG. 2).

Then, control device 230 determines whether predetermined period T2 elapses since the irradiation of surgical field 135 with infrared excitation light 300 in step S204 is started (S210). Control device 230 repeats the pieces of processing in step S206 and S208 in a predetermined period (for example, 1/60 second) until period T2 elapses (NO in S210).

After period T2 elapses (YES in S210), control device 230 causes infrared excitation light source 250 to stop the irradiation of surgical field 135 with infrared excitation light 300, and causes projector 220 to delete projection image G320 (to stop the projection) (S212). Control device 230 returns to the processing in step S200 after the processing in step S212.

Because TOF sensor 260 detects the distance using the infrared light, other light sources are stopped in step S212 before the return to step S200 so as not to have an influence on the detection of the distance. When the light source having no infrared light component is used as projector 220, because projector 220 does not have the influence on the detection of the distance, only infrared excitation light 300 emitted from infrared excitation light source 250 may be stopped in step S212.

The warning is issued through the processing in step S214 when imaging and irradiation device 200 is outside the use height acceptable range, so that the user such as the doctor can recognize that imaging and irradiation device 200 is outside the use height acceptable range. The distance detection processing in step S200 is performed after the processing in step S212, the contradictory control is performed on the TOF sensor 260 and the projector 220, whereby the distance is detected without generating a malfunction of surgery support system 100.

### 7-2-2. Contradictory Control

The contradictory control performed in monitoring the use height of imaging and irradiation device 200 will be described in detail below with reference to FIGS. 14 to 16. FIG. 16 is a timing chart illustrating the operations of infrared excitation light source 250, TOF sensor 260, and visible-light laser 222 according to a height determination result. A horizontal axis in FIG. 16 indicates a time axis. In FIG. 16, a low level in each chart indicates turn-off state, and a high level indicates a lighting state.

As used herein, the “lighting state” means a power-on state of each of infrared excitation light source 250, TOF sensor 260, and visible-light laser 222. On the other hand, the “turn-off state” means a power-off state of each of infrared excitation light source 250, TOF sensor 260, and visible-light laser 222.

During the operation of surgery support system 100, control device 230 periodically makes the height (distance) determination using TOF sensor 260. Specifically, as illustrated in FIG. 16, the determination processing in steps S200 and S202 or steps S216 and S218 in FIG. 14 is performed in period T1 between times t1 and t2, period T1 between times t3 and t4, period T1 between times t5 and t6, period T1 between times t7 and t8, period T1 between times t9 and t10, . . . . At this point, TOF sensor 260 is in the lighting state in which TOF sensor 260 emits infrared detection light 330. In each period T1 in which TOF sensor 260 makes the height determination, control device 230 controls infrared excitation light source 250 and visible-light laser 222 such that infrared excitation light source 250 and visible-light laser 222 are in the turn-off state. That is, the contradictory control is performed such that infrared excitation light source 250 and visible-light laser 222 are put into the turn-off state while TOF sensor 260 is in the lighting state. For example, each of period T1 between times t1 and t2, period T1 between times t3 and t4, period T1 between times t5 and t6, period T1 between times t7 and t8, and period T1 between times t9 and t10 is, for example, a short time of 10 ms to 100 ms, and hardly perceived by a human. Accordingly, even if the contradictory control is performed in the height determination period, the projection image can be perceived by a human so as to be continuously displayed in visible laser beam 320.

In period T1 between times t1 and t2, it is assumed that distance di indicated by the detection result of TOF sensor 260 falls within first interval r1 (=1000 mm±300 mm) indicating the height acceptable range as illustrated in FIG. 15A. At this point, control device 230 determines that detected distance di is in the “normal state” as the height determination result in step S202 of FIG. 14. Control device 230 puts both infrared excitation light source 250 and visible-light laser 222 into the lighting state in subsequent period T2 between times t2 and t3. Therefore, in period T2 between times t2 and t3, projection image G320 can usually be projected onto affected part 140 to normally support the surgery.

In period T1 between times t3 and t4, it is assumed that, as illustrated in FIG. 15B, di′ is outside the range of height acceptable range r1 (=1000 mm±300 mm) while detected as distance di indicated by the detection result of TOF sensor 260. At this point, control device 230 determines that detected distance di is in the “abnormal state” as the height determination result in step S202. In this case, because there is a risk of incorrectly projecting the projection image, it is considered that the surgery support is stopped from the viewpoint of safety. Control device 230 maintains both infrared excitation light source 250 and visible-light laser 222 in the turn-off state in subsequent period T2 between times t4 and t5. Therefore, in period T2 between times t4 and t5, the projection image that is possibly incorrect is not displayed, but a higher priority can be given to the safety to stop the surgery support.

In subsequent period T1 between times t5 and t6, distance di indicated by the detection result of TOF sensor 260 exists between distance d1 at one end of first interval r1 and distance d2 at one end of second interval r2 (for example, 1250 mm). In this case, although imaging and irradiation device 200 exists within the height acceptable range, possibly imaging and irradiation device 200 is instantaneously outside the height acceptable range because imaging and irradiation device 200 is located near a limit of the height acceptable range. For this reason, a hysteresis width is provided at second interval r2 shorter than first interval r1 to perform the determination processing in step S218 of FIG. 14. Therefore, in the case that distance di exists between first distance d1 and distance d2, distance di is determined to be the “abnormal state”, and the safety can be assured in period T2 between times t6 and t7 by performing the operation similar to that in period T2 between times t4 and t5.

In period T1 between times t7 and t8, it is assumed that distance di indicated by the detection result of TOF sensor 260 falls within second interval r2 (=1000 mm±200 mm) at which the hysteresis width is provided in the height acceptable range. At this point, control device 230 determines that detected distance di is in the “normal state” as the height determination result in step S218. Control device 230 puts both infrared excitation light source 250 and visible-light laser 222 into the lighting state in subsequent period T2 between times t8 and t9. Therefore, in period T2 between times t8 and t9, projection image G320 can usually be projected onto affected part 140 to support the surgery again.

A margin period in which infrared excitation light source 250, visible-light laser 222, and TOF sensor 260 become the turn-off state may be provided at the switching times t1, t3, t5, t7, t9, . . . . Therefore, the false detection of infrared detection light 330 can be restrained at the switching time. The margin period may be provided at the switching times t2, t4, t6, t8, t10, . . . .

### 7-3. Effect and the Like

As described above, in the first exemplary embodiment, surgery support system 100 includes infrared camera 210, projector 220, TOF sensor 260, and control device 230. Infrared camera 210 captures an image of affected part 140. Based on the captured image of affected part 140, projector 220 generates projection image G320, and projects projection image G320 onto affected part 140. TOF sensor 260 detects the distance to affected part 140. Control device 230 controls the operations of infrared camera 210 and projector 220. Control device 230 determines whether the distance detected by TOF sensor 260 exists in first interval r1. When the distance detected by TOF sensor 260 exists in first interval r1, control device 230 generates projection image G320, and projects projection image G320 onto affected part 140.

In surgery support system 100, even if the imaging position of affected part 140 is changed, projection image G320 is generated and projected onto affected part 140 when the distance detected by TOF sensor 260 falls within first interval r1, so that the safety can be assured in projecting projection image G320.

Control device 230 issues a predetermined warning when the distance detected by TOF sensor 260 is outside first interval r1.

Surgery support system 100 makes the doctor notice that the distance to affected part 140 runs over first interval r1, so that safety can be secured during the use of surgery support system 100. Therefore, surgery support system 100 can more easily be used by the user such as the doctor.

When the distance detected by TOF sensor 260 is outside first interval r1, projection image G320 may not be projected onto affected part 140 instead of or in addition to the predetermined warning. Therefore, in surgery support system 100, the projection of projection image G320, which is possibly incorrect because the distance runs over first interval r1, can be stopped to improve the safety during the surgery.

Infrared camera 210 may receive infrared fluorescence 310 having the first spectrum, and capture the image of affected part 140. TOF sensor 260 may emit infrared detection light 330 having the second spectrum, and detect the distance to affected part 140. In this case, TOF sensor 260 emits infrared detection light 330 in first period T1, and does not emit infrared detection light 330 in second period T2 different from first period T1. Control device 230 does not project projection image G320 in first period T1, but projects projection image G320 in second period T2. Therefore, the distance to affected part 140 can be detected without generating the malfunction in surgery support system 100.

In the first exemplary embodiment, when the distance is determined to be the “abnormal state”, both infrared excitation light source 250 and visible-light laser 222 are put into the turn-off state, but the present disclosure is not limited thereto. Alternatively, one of infrared excitation light source 250 and visible-light laser 222 may be put into the turn-off state. In the case that infrared excitation light source 250 is put into the turn-off state, ICG infrared fluorescence 310 is not emitted. Therefore, control device 230 can hardly specify the region of affected part 140, and visible laser beam 320 is not emitted even if visible-light laser 222 is in the lighting state. In the case that visible-light laser 222 is put into the turn-off state, originally visible laser beam 320 is not emitted.

When it is determined that the distance is in the “abnormal state”, control device 230 may control infrared camera 210 such that infrared camera 210 does not capture the image instead of or in addition to the control of infrared excitation light source 250 and visible-light laser 222. Control device 230 may control MEMS mirror 221 such that MEMS mirror 221 does not generate projection image G320. That is, control device 230 may control any of the components of surgery support system 100 such that projection image G320 is not projected based on the imaging result of infrared camera 210.

In the first exemplary embodiment, when the distance is determined to be the “abnormal state”, the warning operation is performed such that the message or warning sound indicating that the use height is in the “abnormal state” is generated from the speaker, but the warning operation is not limited thereto. Alternatively, the warning may be the operation to output the information indicating that the distance to the object such as affected part 140 is outside a predetermined interval range. For example, when the distance is determined to be the “abnormal state”, the user such as the doctor may be informed of the “abnormal state”. As to the method for informing the user of the “abnormal state”, visible-light laser 222 is switched to a visible-light laser having another wavelength, and visible laser beam 320 may be emitted while the color of visible laser beam 320 is changed. Alternatively, the warning may be issued by projecting the projection image including a text message indicating the “abnormal state”.

In the first exemplary embodiment, in issuing the warning, projection image G320 is deleted based on the imaging result of infrared camera 210, but the present disclosure is not limited thereto. Alternatively, for example, the projection image may be used as the warning based on the imaging result of infrared camera 210. For example, based on the imaging result of infrared camera 210, the projection image may be projected while the color of the projection image is changed.

In the first exemplary embodiment, infrared detection light 330 having the spectrum superimposed on the spectrum of infrared fluorescence 310 is used in TOF sensor 260, but the present disclosure is not limited thereto. Alternatively, for example, TOF sensor 260 may detect the distance by emitting the detection light having the spectrum that is not superimposed on the spectrum of infrared fluorescence 310. In this case, for example, a wavelength filter cutting the spectrum of infrared fluorescence 310 may be provided in the light emitting unit of TOF sensor 260. At this point, in the wavelength band of the wavelengths of 850 nm to 950 nm, an air transmittance is large, and the distance is easily detected. Therefore, the contradictory control is performed with no use of the wavelength filter, which allows the improvement of distance detection efficiency.

In the first exemplary embodiment, the use height is monitored by the distance detected by TOF sensor 260, but the present disclosure is not limited thereto. Alternatively, for example, the distance between imaging and irradiation device 200 and the object such as affected part 140 may be monitored by the distance detected by TOF sensor 260 such that surgery support system 100 can properly be operated even if the orientation of imaging and irradiation device 200 is changed.

In the description with reference to FIG. 16, a state transition, namely, the “lighting state” and the “turn-off state” of infrared excitation light source 250 and visible-light laser 222 are performed by switching between the power-on state and power-off state of the light source, but the present disclosure is not limited thereto. Alternatively, the “lighting state” and the “turn-off state” may be performed by switching between a light shielding on state and a light shielding off state of a light shielding unit even if the light source is maintained in the power-on state.

### Other Exemplary Embodiments

The first exemplary embodiment is described above as a technical illustration of the present disclosure. However, the technology of the present disclosure is not limited to the first exemplary embodiment, but the technology of the present disclosure can be applied to exemplary embodiments in which changes, replacements, additions, and omissions are properly made. A new exemplary embodiment can be made by a combination of the components described in the first exemplary embodiment.

Other exemplary embodiments will be described below.

In the first exemplary embodiment, by way of example, the present disclosure is applied to the medical use such as the surgery, but the present disclosure is not limited thereto. Alternatively, for example, the present disclosure can be applied to the cases, such as a construction site, a mining site, a building site, and a material processing factory, in which the work is performed on a target object whose state change cannot visually be recognized.

Specifically, in the construction site, the mining site, the building site, and the material processing factory instead of the medical instrument of the first exemplary embodiment, a fluorescent material is applied, kneaded, or flown in the target object whose state change cannot visually be recognized, and the imaging target of infrared camera 210 may be formed. A heat generation place is detected by not the light emitting sensor but a thermal sensor, only the heat generation place or the boundary may be scanned.

In the first exemplary embodiment, the laser source is used by way of example. The projection of the cutting auxiliary line or the surgical auxiliary information is not limited to the laser source. That is, the cutting auxiliary line and the surgical auxiliary information may be projected using a light source other than the laser source.

In the first exemplary embodiment, the cutting auxiliary line or the surgical auxiliary information is projected using identical visible-light laser 222 that projects the region specified as the affected part, but the present disclosure is not limited thereto. Alternatively, the cutting auxiliary line or the surgical auxiliary information may be projected using a light source different from visible-light laser 222 that projects the region specified as the affected part. However, the control is performed such that the projection is performed according to the irradiation of the region specified as the affected part.

In the first exemplary embodiment, TOF sensor 260 is used as the distance detector, but the present disclosure is not limited thereto. Alternatively, for example, a sensor may emit the infrared detection light having a well-known pattern like a random dot pattern, and measure the distance based on a pattern deviation of the reflected light. In this case, the distance detector can detect the distance information as a distance image expressing the distance at each dot in the two-dimensional region.

In the first exemplary embodiment, projection image G320 is illustrated as the monochrome, uniform image in visible-light laser 222. The projection image projected by the projector is not limited to the monochrome, uniform image, but a gray-scaled projection image or a full-color projection image may be projected, or any image may be projected.

The exemplary embodiments are described above as the technical illustration of the present disclosure. The accompanying drawings and the detailed description are provided in the exemplary embodiments.

In the components described in the accompanying drawings and the detailed description, not only the component necessary for solving the problem but also the component that is not necessary for solving the problem can be included in order to illustrate the above technology. Therefore, it is noted that the unnecessary component should not directly be recognized as the necessary component even if the unnecessary component is described in the accompanying drawings and the detailed description.

Because the exemplary embodiments are used to illustrate the technology of the present disclosure, various changes, replacements, additions, omissions, and the like can be made in claims or a range equivalent to claims.

For example, the projection system of the present disclosure can be applied to the cases, such as medical use, the construction site, the mining site, a building site, and the material processing factory, in which the work is performed on the target object whose state change cannot visually be recognized.

What is claimed is:

1. A projection system comprising:
an imaging unit that captures an image of an object;
a projector that generates a projection image based on the captured image to project the projection image onto the object;
a distance detector that detects a distance to the object; and
a controller that controls operations of the imaging unit and the projector,
wherein the controller determines whether the distance detected by the distance detector falls within a predetermined range, and
the controller causes the projector to project the projection image onto the object based on the captured image when the detected distance falls within the predetermined range.

2. The projection system according to claim 1, wherein the controller issues a predetermined warning when the detected distance does not fall within the predetermined range.

3. The projection system according to claim 1, wherein the controller does not cause the projector to project the projection image onto the object when the detected distance does not fall within the predetermined range.

4. The projection system according to claim 1, wherein the imaging unit receives light having a first spectrum to capture the image of the object,
the distance detector emits detection light having a second spectrum to detect the distance to the object,
the distance detector emits the detection light in a first period, but does not emit the detection light in a second period different from the first period, and
the controller does not cause the projector to project the projection image in the first period, but causes the projector to project the projection image in the second period.

5. The projection system according to claim 4, wherein
the first period and the second period are alternately repeated, and
when the distance detected by the distance detector does not fall within the predetermined range in the first period, the controller does not cause the projector to project the projection image based on the captured image in the second period subsequent to the first period.

6. The projection system according to claim 4, further comprising an excitation light source that irradiates the object with excitation light corresponding to the first spectrum,
wherein the object includes a photosensitive substance that reacts with the excitation light to emit the light having the first spectrum.

7. The projection system according to claim 6, wherein the controller controls the excitation light source such that the excitation light source does not emit the excitation light in the first period, and emits the excitation light in the second period.

8. The projection system according to claim 2, wherein the warning is issued by outputting information indicating that the distance to the object does not fall within the predetermined range.

9. The projection system according to claim 1, wherein the object is an affected part of a living body.
